(19) United States
(12) Patent Application Publication
(10) Pub. No.: US 2024/0312561 A1
(43) Pub. Date: Sep. 19, 2024

(54) OPTIMIZATION OF SEQUENCING PANEL ASSIGNMENTS

(71) Applicant: GRAIL, LLC, Menlo Park, CA (US)

(72) Inventors: Robert Abe Paine Calef, Redwood City, CA (US); Alexander P. Fields, Burlingame, CA (US); Samuel S. Gross, Sunnyvale, CA (US)

(21) Appl. No.: 18/603,727

(22) Filed: Mar. 13, 2024

Related U.S. Application Data
(60) Provisional application No. 63/489,826, filed on Mar. 13, 2023.

(51) Int. Cl. G16B 20/20; C12Q 1/6809; C12Q 1/6886

(52) U.S. Cl. CPC G16B 20/20; C12Q 1/6809; C12Q 1/6886

(57) ABSTRACT

The present disclosure relates to a method for improving sequencing panel assignments for samples from two or more individual. The system is configured to generate a sequencing panel assignment having an optimized set of samples for each panel that reduces sequencing costs but does not compromise Limit of Detection of the assay.

### CROSS-REFERENCE TO RELATED APPLICATIONS

The present application claims the benefit of and priority to U.S. Provisional Application No. 63/489,826 filed on Mar. 13, 2023, which is incorporated by reference.

### BACKGROUND

Cancer is a leading cause of death worldwide. The fatality of cancer is heightened by the fact that cancer is usually detected in latter stages, limiting efficacy of treatment options for long-term survival. Current detection methods generally are cancer type specific, i.e., each cancer type is individually screened for. Each individual screening process is tailored to the cancer type. For example, mammography scans are utilized in breast cancer detection, whereas colonoscopy or fecal tests have helped with colorectal cancer detection. Each varied screening method is not cross-applicable to other cancer types. For example, to screen one individual for three different possible cancer types, a healthcare provider would need to perform or order to be performed three different screening processes. Each of those screening processes may entail a combination of invasive and/or non-invasive procedures to identify tumorous growths, collect a biopsy of the growth, and perform analysis on the tissue biopsy.

Furthermore, present screening methods are encumbered by low detection rates or high false positive rates. Low detection rates often fail to detect early-stage cancers as the cancers are just developing. A high positive rate misdiagnoses cancer-free individuals as positive for cancer status. As a result, most screening tests are only practical when they are used to test individuals who have a high risk of developing the screened cancer, and they have limited ability to detect cancers in the general population.

Novel research has implicated various genetic variations as early markers and indicators of cancer development. For example, such research has focused on copy number variation, small variants (including single nucleotide polymorphisms, insertions, and deletions), aberrant methylation, etc. This research employs models to identify correlations between these genetic variations and cancer status. Nonetheless, even such models face a number of challenges. Early cancer detection is particularly challenging due to the miniscule ratio of tumor cells to non-cancer cells in the subject. The miniscule ratio may be on the order of 1:1000, 1:10,000, or even 1:100,000. This creates a challenge of detecting small amounts of cancer signal amidst healthy signal.

To conduct a survey of tumor fractions across cfDNA samples, targeted sequencing panels (e.g., to detect small nucleotide variants) are designed to identify somatic variants called from whole-genome sequencing of each participant's tumor biopsy. Rather than ordering one panel per participant, the sequencing data from multiple participants can be combined into a single panel, both to reduce cost and simplify operations. A challenge arises in optimizing the combination of multiple participants into a single panel. A rudimentary approach would randomly assign participants to panels, but such approach could pair nonoptimal participants, e.g., participants having different number of variants (which would lead to less than closest packing, i.e., more panels than necessary), participants having variants with probes of different pull-down efficiency (which would create high variability in variant coverages, leading to decreased sensitivity in low-coverage variants), or participants having variants with differing noise rates (which, likewise, would create varying sensitivity of the panel, leading to potential missed variant calls).

The present disclosure is directed to addressing the above-referenced challenges, in particular optimizing assignment of participants to targeted panels. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The invention(s) described herein this disclosure provide for improvements to cancer detection and treatment, in particular, optimizing assignment of participants to targeted panels. In particular, a panel assignment model takes into consideration various characteristics of participants, characteristics of variants to be screened for each participant, characteristics of probes targeting the variants, or some combination thereof in determining assignment of participants to panels and in determining variants to target screening in each participant. Such optimization improves the targeted sequencing process, e.g., by improving sensitivity of variant calling among participants in a single panel, and by identifying a close-packing configuration of participants to panels thereby minimizing number of panels and sequencing costs associate therewith.

The invention(s) comprise screening for cancer signal in a cell-free deoxyribonucleic acid (cfDNA) sample of a subject. Such cfDNA samples may comprise thousands, tens of thousands, hundreds of thousands, millions, or more of cfDNA fragments, thereby resulting in a similar order of sequence reads output by a sequencer, or even a multiple of such order based on a sequencing depth of the sample. Each sequence read relating to cfDNA fragments can vary in length, e.g., up to 250, 300, 350, 400, 450, 500, 550, 600, 650, 700, 750, 800, 850, 900, 950, or 1000 bp in length. These next-generation sequencing techniques greatly increase volume of fragments that can be sequenced and analyzed, thereby enabling such models to identify even miniscule amounts of cancer signal in a sample. The invention(s) are capable of screening for cancer generally, or for a plurality of cancer types from a single sample. This improves over conventional screening methods tailored per cancer type by providing a single comprehensive screening that is capable of screening a variety of cancer types from a single cfDNA sample. For example, screening for different cancers generally involved separate screening processes that would each detect abnormal tissue growths, collect tissue biopsies of the detected growths, and perform laboratory analysis of the collected biopsies to assess malignancy.

The invention(s) implement computer models to identify and quantify the cancer signal. In one or more embodiments, the computer models may perform variant calling as features to cancer classification. The computers models may include trained cancer classifiers configured to input a feature vector generated based on the called variants and to output a cancer prediction based on the input feature vector. The cancer prediction may be a binary prediction and/or a multiclass prediction. The binary prediction may be a likelihood of presence of cancer. The multiclass prediction may be a likelihood of a particular cancer type from a plurality of cancer types evaluated. Training a cancer classifier capable of screening between a plurality of cancer types enables medical care professionals to utilize a single comprehensive screening rather than multiple disparate screenings. In one or more embodiments, the cancer classifier is a machine-learning model rooted in computer functionality and not practically performable in the human mind. In one or more embodiments, the cancer classifier includes non-mathematical operations, including operations based in the manipulation of electronic data in the context of a computing device.

The cancer prediction can be used by a healthcare provider for diagnosis, prognosis, tailoring treatment, evaluation of treatment, minimum residual disease detection, etc. This insight from minimally-invasive liquid biopsies can provide for increased screening frequency, whilst minimizing harm to the patient.

Clause 1. A method for performing targeted variant sequencing of a set of samples, the method comprising: obtaining initial sequencing data for each sample describing presence or absence of each of a plurality of genetic variants in a reference genome, wherein the initial sequencing data comprises sequence reads of nucleic acid fragments in a biological sample obtained from a subject; determining, for each sample, a number of genetic variants present in the sequencing data of the sample; determining, for each sample, one or more characteristics for each genetic variant present in the sequencing data of the sample; applying a panel assignment model to determine a panel assignment for each sample to one of a plurality of targeted variant sequencing panels, wherein applying the panel assignment model comprises: iterating through each sample in the set of samples and to determine the corresponding panel assignment that optimizes uniformity of panel size across the targeted variant sequencing panels and uniformity of characteristics of the genetic variants across samples assigned to each targeted variant sequencing panel, performing a swapping operation to swap panel assignments of at least two samples to further optimize the uniformity of panel size across the targeted variant sequencing panels and the uniformity of characteristics of the genetic variants across samples assigned to each targeted variant sequencing panel; and generating each targeted sequencing panel inclusive of samples with panel assignments to the targeted sequencing panel and indicating an aggregate set of genetic variants across the samples assigned to the targeted sequencing panel; and performing the targeted variant sequencing with each targeted variant sequencing panel comprising the assigned samples on cell-free deoxyribonucleic acid (cfDNA) samples subsequently collected from the subjects.

Clause 2. The method of clause 1 or any clause dependent thereon, wherein the initial sequencing includes whole genome sequencing or whole exome sequencing of each sample.

Clause 3. The method of clause 1 or any clause dependent thereon, wherein the genetic variants include single nucleotide variants, insertion variants, and deletion variants.

Clause 4. The method of clause 1 or any clause dependent thereon, wherein the genetic variants include copy number variants.

Clause 5. The method of clause 1 or any clause dependent thereon, wherein the one or more characteristics for each genetic variant are selected from a group consisting of: a guanine and cytosine content of the genetic variant, an error rate of targeting probes of the genetic variant, a sequencing depth count of the genetic variant, a presence or absence of the genetic variant, a mean allele frequency of the genetic variant, a total number of genetic variants, and an allele frequency of true genetic variants.

Clause 6. The method of clause 1 or any clause dependent thereon, wherein applying the panel assignment model further comprises: determining the panel assignment for each sample in the set of samples according to one or more hard constraints.

Clause 7. The method of clause 6, wherein the one or more hard constraints are selected from a group consisting of: a maximum number of samples per targeted sequencing panel, a maximum number of a samples of one type per targeted sequencing panel, or a total number of genetic variants per targeted sequencing panel.

Clause 8. The method of clause 1 or any clause dependent thereon, wherein the panel assignment model comprises a function scoring the uniformity of panel size across the targeted variant sequencing panels and the uniformity of characteristics of the genetic variants across samples assigned to each targeted variant sequencing panel.

Clause 9. The method of clause 8, wherein iterating through each sample in the set of samples to determine the corresponding panel assignment comprises: applying a greedy algorithm or a dynamic programming algorithm to the function to determine the corresponding panel assignment.

Clause 10. The method of clause 8 or any clause dependent thereon, wherein performing a swapping operation comprises: evaluating a change in score based on the function evaluating the swap of the at least two samples; and swapping the panel assignments of the at least two samples based on the change in score being above a threshold.

Clause 11. The method of clause 1 or any clause dependent thereon, wherein the swapping operation is performed iteratively to assess pairs of samples assigned to different targeted sequencing panels.

Clause 12. The method of clause 1 or any clause dependent thereon, further comprising: for each targeted sequencing panel, identifying an optimal aggregate set of genetic variants across the samples based on the characteristics of the genetic variants in each sample assigned to the targeted sequencing panel.

Clause 13. The method of clause 12 or any clause dependent thereon, wherein identifying the optimal set of genetic variants for each targeted sequencing panel comprises, for each targeted sequencing panel: determining whether each sample has the total number of genetic variants above a per-sample threshold; and responsive to determining at least one sample has the total number of genetic variants above the per-sample threshold, identifying a subset of genetic variants in the sample to include in the targeted sequencing panel that optimizes the characteristics of the optimal aggregate set of genetic variants.

Clause 14. The method of clause 13, wherein identifying the subset of genetic variants to include in the targeted sequencing panel comprises inclusion of genetic variants present in other samples of the targeted sequencing panel.

Clause 15. The method of clause 1 or any clause dependent thereon, wherein generating each targeted sequencing panel further comprises identifying corresponding targeting probes to target the aggregate set of genetic variants.

Clause 16. The method of clause 1 or any clause dependent thereon, the method further comprising: obtaining targeted sequencing data for the set of samples from the targeted variant sequencing panels on the cfDNA samples, wherein the targeted sequencing data comprises sequence reads of cell-free nucleic acid fragments in a blood sample; for each cfDNA sample: calling one or more variants present in the targeted sequencing data; determining a feature vector based on the called one or more variants; and applying a cancer classifier to the feature vector to predict a tumor fraction in the cfDNA sample.

Clause 17. A non-transitory computer-readable storage medium storing instructions that, when executed by a processor, cause the process to perform the method of clause 1 or any clause dependent thereon.

Clause 18. A system comprising: a processor; and the non-transitory computer-readable storage medium of clause 17.

Clause 19. A targeted sequencing panel comprising: a set of targeting probes to target an aggregate set of genetic variants across a plurality of samples assigned to the targeted sequencing panel, wherein the aggregate set of genetic variants and the plurality of samples are determined by: obtaining initial sequencing data for each sample in a set of samples describing presence or absence of each of a plurality of genetic variants in a reference genome, wherein the initial sequencing data comprises sequence reads of nucleic acid fragments in a biological sample obtained from one subject; determining, for each sample, a number of genetic variants present in the sequencing data of the sample; determining, for each sample, one or more characteristics for each genetic variant present in the sequencing data of the sample; applying a panel assignment model to determine a panel assignment for each sample to one of a plurality of targeted variant sequencing panels, wherein applying the panel assignment model comprises: iterating through each sample in the set of samples and to determine the corresponding panel assignment that optimizes uniformity of panel size across the targeted variant sequencing panels and uniformity of characteristics of the genetic variants across samples assigned to each targeted variant sequencing panel, performing a swapping operation to swap panel assignments of at least two samples to further optimize the uniformity of panel size across the targeted variant sequencing panels and the uniformity of characteristics of the genetic variants across samples assigned to each targeted variant sequencing panel.

Clause 20. A plurality of targeted sequencing panels comprising: for each targeted sequencing panel, a set of targeting probes to target an aggregate set of genetic variants across a plurality of samples assigned to the targeted sequencing panel, wherein the targeted sequencing panels are determined by: obtaining initial sequencing data for each sample in a set of samples describing presence or absence of each of a plurality of genetic variants in a reference genome, wherein the initial sequencing data comprises sequence reads of nucleic acid fragments in a biological sample obtained from one subject; determining, for each sample, a number of genetic variants present in the sequencing data of the sample; determining, for each sample, one or more characteristics for each genetic variant present in the sequencing data of the sample; applying a panel assignment model to determine a panel assignment for each sample to one of a plurality of targeted variant sequencing panels, wherein applying the panel assignment model comprises: iterating through each sample in the set of samples and to determine the corresponding panel assignment that optimizes uniformity of panel size across the targeted variant sequencing panels and uniformity of characteristics of the genetic variants across samples assigned to each targeted variant sequencing panel, performing a swapping operation to swap panel assignments of at least two samples to further optimize the uniformity of panel size across the targeted variant sequencing panels and the uniformity of characteristics of the genetic variants across samples assigned to each targeted variant sequencing panel.

Clause 21. A method for improving sequencing panel assignment for samples from two or more individuals, the method comprising: retrieving sequencing data for each sample; selecting a feature value from the sequencing data; applying a machine learning model that determines the sequencing panel assignment based on the feature values from the sequencing data; and generating an optimized sequencing panel assignment comprising samples from two or more individuals.

Clause 22. The method of clause 21, wherein the selecting step further comprises determining a set of optimized feature values.

Clause 23. The method of clause 22, wherein the machine learning model is selected from: a classifier model, a pre-specified algorithm, and a regression model.

Clause 24. The method of clause 23, wherein the machine learning model is a classifier model.

Clause 25. The method of any one of clauses 21-24, wherein applying the classifier model comprises: ranking the samples based on decreasing feature value; applying a greedy algorithm to add a next-highest ranked sample of the remaining ranked samples to a panel, wherein the panel to which the sample is sorted comprises the lowest value of feature values.

Clause 26. The method of clause 25, further comprising: iterating through samples to assign the samples to a panel; determining mean of feature values for each panel; swapping two samples between two different panels; and measuring deviation of mean feature value for each of the two different panels following the swap.

Clause 27. The method of clause 26, further comprising repeating the steps of claim 26 for a pre-specified number of swaps, thereby generating a panel assignment based on the feature values from the sequencing data.

Clause 28. The method of clause 27, wherein the repeating step is performed until a reduction in the mean feature value is below a threshold.

Clause 29. The method of any one of clauses 21-28, wherein the panel comprises 16 samples.

Clause 30. The method of any one of clauses 21-28, wherein the panel has no more than 16 samples.

Clause 31. The method of any one of clauses 21-30, wherein the panel comprises a benchmarking sample.

Clause 32. The method of clause 31, wherein the panel has no more than one benchmarking sample.

Clause 33. The method of clause 32, wherein applying the classifier model comprises: seeding the sequencing panel based on decreasing number of feature values; swapping sequencing panel assignments for two participants seeded in two different panels; measuring reduction in loss function after swapping; and comparing the set of sequencing panel assignments and the feature values.

Clause 34. The method of clause 33, further comprising repeating for a pre-specified number of steps or until the reduction in the loss function is below a threshold.

Clause 35. The method of clause 33 or 34, further comprising: determining the

sequencing panel assignments that meet DRAWING.

Clause 36. The method of any one of clauses 21-35, wherein the sequencing data

is obtained from sequencing cell-free nucleic acid molecules existing in biological samples obtained from a plurality of individuals.

Clause 37. The method of any of clauses 21-36, wherein the feature values correspond to a genomic region comprising one or more of the following: cancer-related genes, mutation hotspots, and viral regions.

Clause 38. The method of any one of clauses 21-37, wherein the sequencing data comprises genomic regions associated with a high signal cancer or a liquid cancer.

Clause 39. The method of any one of clauses 21-38, wherein the feature values represent features corresponding to one or more of the following: a GC content, an error rate, a sequencing depth count, a presence or absence of a variant, a mean allele frequency, a total number of small variants, and an allele frequency of true variants.

Clause 40. The method of any one of clauses 21-39, wherein the feature value is a sum of a plurality of feature values.

Clause 41. The method of any one of clauses 21-40, wherein the feature value is a variant.

Clause 42. The method of clause 41, wherein the variant comprises one or more of the following: a single nucleotide variant, an insertion, and a deletion.

Clause 43. A non-transitory computer-readable medium storing one or more programs, the one or more programs including instructions which, when executed by an electronic device including a processor, cause the device to perform the method of any one of clauses 21-42.

Clause 44. An electronic device, comprising: one or more processors; memory; and one or more programs, wherein the one or more programs are stored in the memory and configured to be executed by the one or more processors, the one or more programs including instructions for performing the method of any one of clauses 101-42.

### BRIEF DESCRIPTION OF DRAWINGS

FIG. 1 is an exemplary flowchart describing an overall workflow of cancer classification of a sample, according to one or more embodiments.

FIG. 2A illustrates an exemplary flowchart of devices for sequencing nucleic acid samples according to one or more embodiments.

FIG. 2B is an exemplary block diagram of an analytics system, according to one or more embodiments.

FIG. 3 is a flowchart of sequencing a nucleic acid sample, according to one or more embodiments.

FIG. 4 is a flowchart of optimized sequencing panel assignment, according to one or more embodiments.

FIG. 5 is flowchart of a variant calling method, according to one or more embodiments.

FIG. 6A is a flowchart of training a cancer classifier based on variant features, according to one or more embodiments.

FIG. 6B is a flowchart of deploying a cancer classifier based on variant features, according to one or more embodiments.

FIG. 7 illustrates a tumor fraction (TF) plot for 210 participants.

FIG. 8 illustrates a tumor fraction (TF) plot for 201 benchmarking samples.

FIG. 9 illustrates a tumor fraction (TF) plot for 5 of the 201 benchmarking samples having a 10% TF threshold shown also in FIG. 8.

FIG. 10 shows characteristics including number of variants for 5 benchmarking samples as shown in FIG. 8 and FIG. 9.

FIG. 11 shows a histogram for error rate (y-axis) for each of the different conversion types (x-axis) when analyzing SNPs.

FIG. 12 shows a bar graph for collapsed mean target coverage (MTC) versus input cfDNA yield.

FIG. 13 shows a dot plot for MTC versus input cfDNA mass.

FIG. 14 shoes a box plot for collapsed MTC for each of the indicated conditions on the X-axis.

FIG. 15 shows a graph for Limit of Detection (LoD) for total depth from samples taken from a single tube of plasma.

FIG. 16 shows a dot plot for Limit of Detection (LoD) modelling based on logMin tumor fraction (TF) (x-axis) versus logNvariants (y-axis).

FIG. 17 shows a graph for LoD modelling based on LogMinTF (x-axis) versus Fraction of participants (y-axis).

FIG. 18 shows a dot plot for LoD modelling based on LogMinTF (x-axis) and LogNVariants (y-axis).

FIG. 19 shows a graph for LoD modelling based on LogMinTF (x-axis) and Fraction of participants (y-axis).

FIG. 20 shows a bar graph of the types and number of cancers present in the 129 samples.

FIG. 21 shows a bar graph of the stages of the cancer types present in the 129 samples.

FIG. 22 show plot of the Log-likelihood ratio (LLR) for a true call versus noise.

FIG. 23 shows a histogram for error rate (y-axis) for each of the different conversion types (x-axis) when analyzing SNPs.

FIG. 24 illustrates dot plots for combinations of samples showing relationship between GC content and sequencing depth.

FIG. 25 shows a box plot summarizing the plot from FIG. 24.

FIG. 26 shows a heat map for the LoD framework and idenfitying the priortizaiton of SNPs driven by SNP types (i.e., errorate.)

FIG. 27A shows a dot plot showing GC content (x-axis) verses sequencing depth.

FIG. 27B shows a dot plot showing GC content (x-axis) verses mean_bagsize.

FIG. 28A shows a box plot summarizing normalized depth for each of the Samples corresponding the GC bins on the x-axis.

FIG. 28B shows a table corresponding to the data in FIG. 28A.

FIG. 29 shows a dot plot of total coverage (“depth_raw”; x-axis) versus mean_bagsize (“mean_bagsize_one_tube”; y-axis).

FIG. 30 shows a dot plot of bagsize versus duplex %.

FIG. 31 shows a plot for comparing noise rate (“value”; y-axis) and different conversion types (“snp_group”; x-axis).

FIG. 32A shows a histogram for relative cost (y-axis) and expected effort free unique coverage (EFC) for all samples.

FIG. 32B shows a histogram for relative cost (y-axis) and expected effort free unique coverage (EFC) for all samples.

FIG. 33A and FIG. 33B illustrates the optimal number of panels as a graph (FIG. 33A) and in table format (FIG. 33B).

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

Early detection and classification of cancer is an important technology. Being able to detect cancer before it becomes symptomatic is beneficial to all parties involved, including patients, doctors, and loved ones. For patients, early cancer detection allows them a greater chance of a beneficial outcome; for doctors, early cancer detection allows more pathways of treatment that may lead to a beneficial outcome; for loved ones, early cancer detection increases the likelihood of not losing their friends and family to the disease.

Recently, early cancer detection technology has progressed towards analyzing genetic fragments (e.g., DNA) of a person, e.g., in their blood, to determine if any of those genetic fragments originate from cancer cells. These new techniques allow doctors to identify a cancer presence in a patient that may not be detectable otherwise, e.g., in conventional screening processes. For instance, consider the example of a person at high risk for breast cancer. Traditionally, this person will regularly visit their doctor for a mammogram, which creates an image of their breast tissue (e.g., taking x-ray images) that a doctor uses to identify cancerous tissue. Unfortunately, with even the highest resolution mammograms, doctors are only able to identify tumors once they are approximately a millimeter in size. This means that the cancer has been present for some time in the person and has gone undiagnosed and untreated. Visual determinations like this are typical for most cancers-that is, only being identifiable once it has grown to a sufficient size and has become identifiable with some sort of imaging technology.

Cancer detection using analysis of genetic fragments in a patient's, e.g., blood alleviates this issue. To illustrate, cancer cells will start sloughing DNA fragments into a person's bloodstream as soon as they form. This occurs when there are very few of the cancer cells, and before they would be visible with imaging techniques. With the appropriate methods, therefore, a system that analyzes DNA fragments in the bloodstream could identify cancer presence in a person based on sloughed cancer DNA fragments, and, more importantly, they system could do so before the cancer is identifiable using more traditional cancer detection techniques.

Cancer detection based on the analysis of DNA fragments is enabled by next-generation sequencing (“NGS”) techniques. NGS, broadly, is a group of technologies that allows for high throughput sequencing of genetic material. As discussed in greater detail herein, NGS largely consists of (1) sample preparation, (2) DNA sequencing, and (3) data analysis. Sample preparation is the laboratory methods necessary to prepare DNA fragments for sequencing, sequencing is the process of reading the ordered nucleotides in the samples, and data analysis is processing and analyzing the genetic information in the sequencing data to identify cancer presence.

While these steps of NGS may help enable early cancer detection, they also introduce their own complex, detrimental problems to cancer detection and, therefore, any improvements to sample preparation, DNA sequencing, and/or data analysis, including the pre-processing, algorithmic processing, and summary or presentation of predications or conclusions, results in an improvement to cancer detection technologies and early cancer detection more generally.

To illustrate, as an example, problems introduced in (1) sample preparation include optimizing samples assigned to panels, DNA sample quality, sample contamination, fragmentation bias, and accurate indexing. Remedying these problems would yield better genetic data for cancer detection. Similarly, problems introduced in (2) sequencing include, for example, errors in accurate transcribing of fragments (e.g., reading an “A” instead of a “C”, etc.), incorrect or difficult fragment assembly and overlap, disparate coverage uniformity, sequencing depth vs. cost vs. specificity, and insufficient sequencing length. Again, remedying any of these problems would yield improved genetic data for cancer detection.

The problems in (3) data analysis are the most daunting and complex. The introduced challenges stem from the vast amounts of data created by NGS sequencing techniques. The created genetic datasets are typically on the order of terabytes, and effectively and efficiently analyzing that amount of data is both procedurally and computationally demanding. For instance, analyzing NGS sequencing involves several baseline processing steps such as, e.g., aligning reads to one another, aligning and mapping reads to a reference genome, identifying and calling variant genes, identifying and calling abnormally methylated genes, generating functional annotations, etc. Performing any of these processes on terabytes of genetic data is computationally expensive for even the most powerful of computer architectures, and completely impossible for a normal human mind. Additionally, with the genetic sequencing data derived from the error-prone processes of sample preparation and sequence reading, large portions of the resulting genetic data may be low-quality or unusable for cancer identification.

For example, large amounts of the genetic data may include contaminated samples, transcription errors, mismatched regions, overrepresented regions, etc. and may be unsuitable for high accuracy cancer detection. Identifying and accounting for low quality genetic data across the vast amount of genetic data obtained from NGS sequencing is also procedurally and computationally rigorous to accomplish and is also not practically performable by a human mind. Overall, any process created that leads to more efficient processing of large array sequencing data would be an improvement to cancer detection using NGS sequencing.

Finally, and perhaps most importantly, accurate identification of informative DNA from NGS data to identify a cancer presence is also difficult (much more in the early cancer detection context). To be effective, algorithms are sought to compensate for, e.g., errors generated by sample preparation and sequencing, and to overcome the large-scale data analysis problems accompanying NGS techniques. That is, designing a machine learning model or models, or other computational processing algorithms, that enable early cancer detection based on next generation sequencing techniques must be configured to account for the problems that those techniques create. Some of those techniques and models are discussed hereinbelow and particular improvements to state-of-the-art techniques and models are further discussed.

The training of the machine-learned models described herein (such as the contamination models, the cancer classifier, any other neural network, and any other model referenced herein) include the performance of one or more non-mathematical operations or implementation of non-mathematical functions at least in part by a machine or computing system, examples of which include but are not limited to data loading operations, data storage operations, data toggling or modification operations, non-transitory computer-readable storage medium modification operations, metadata removal or data cleansing operations, data compression operations, protein structure modification operations, image modification operations, noise application operations, noise removal operations, and the like. Accordingly, the training of the machine-learned models described herein may be based on or may involve mathematical concepts, but is not simply limited to the performance of a mathematical calculation, a mathematical operation, or an act of calculating a variable or number using mathematical methods.

Likewise, it should be noted that the training of these models described herein cannot be practically performed in the human mind. The models are innately complex including vast amounts of weights and parameters associated through one or more complex functions. Training and/or deployment of such models involve so great a number of operations that it is not feasibly performable by the human mind alone, nor with the assistance of pen and paper. In such embodiments, the operations may number in the hundreds, thousands, tens of thousands, hundreds of thousands, millions, billions, or trillions. Moreover, the training data may include hundreds, thousands, tens of thousands, hundreds of thousands, millions, or billions of sequence reads, each sequence read may further include anywhere from hundreds up to thousands of nucleotides. Rapid processing of such sheer quantity of sequencing data is also paramount to the efficacy and adoptability in early cancer screening. For example, even if (for the sake of argument) the human mind performed each and every calculation needed, such timespan would likely be on the order of years (if not tens of years), at which point the advantage of sequence data analysis for early cancer detection would be lost. Accordingly, such models are necessarily rooted in computer-technology for their implementation and use.

### I.A. Cancer Classification Workflow

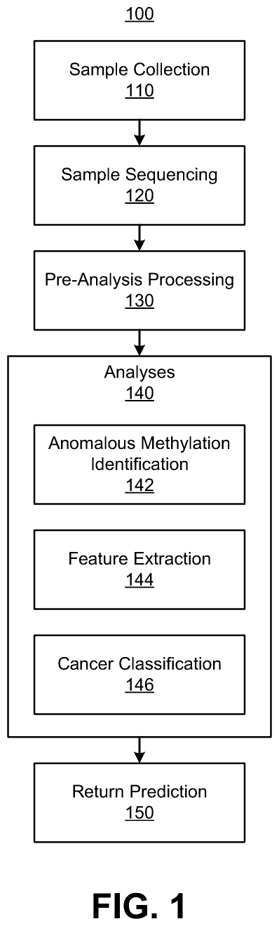

FIG. 1 is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in individuals. From a healthcare standpoint, the workflow 100 can serve to supplement other existing cancer diagnostic tools. The workflow 100 may serve to provide early cancer detection and/or routine cancer monitoring to better inform treatment plans for individuals diagnosed with cancer. The overall workflow 100 may include additional/fewer steps than those shown in FIG. 1.

A healthcare provider performs sample collection 110. An individual to undergo cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. In one or more embodiments, collecting of the sample is minimally invasive or non-invasive. The sample includes genetic material belonging to the individual, which may be extracted and sequenced for cancer classification. Once the sample is collected, the sample is provided to a sequencing device. Along with the sample, the healthcare provider may collect other information relating to the individual, e.g., biological sex, age, race, smoking status, other health metrics, any prior diagnoses, etc.

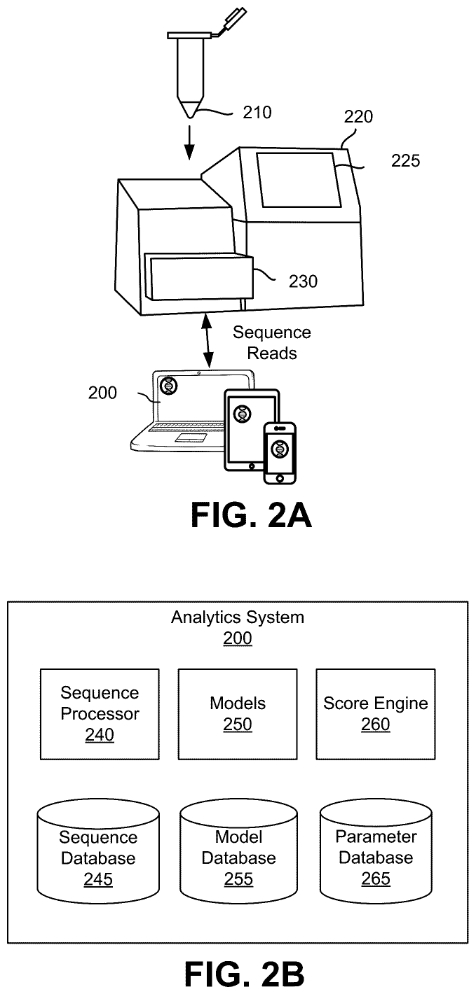

A sequencing device performs sample sequencing 120. A lab clinician may perform one or more processing steps to the sample in preparation of sequencing. Once prepared, the clinician loads the sample in the sequencing device. An example of devices utilizes in sequencing is further described in conjunction with FIGS. 2A & 2B. The sequencing device generally extracts and isolates fragments of nucleic acid that are sequenced to determine a sequence of nucleobases corresponding to the fragments. Sequencing may also include amplification of nucleic material. Different sequencing processes include Sanger sequencing, fragment analysis, and other next-generation sequencing techniques. Next-generation sequencing is capable of yielding high-throughput sequencing data, e.g., 10,000, 100,000, 1,000,000, or 10,000,000 sequence reads, and each sequence read may be of length 50 bp, 100 bp, 150 bp, 200 bp, 250 bp, 300 bp, etc. Accordingly, the high-throughput sequencing data is of a size that it is impractical for a human mind to analyze. Sequencing may be whole-genome sequencing or targeted sequencing with a target panel. In context of DNA methylation, bisulfite sequencing can determine methylations status through bisulfite conversion of unmethylated cytosines at CpG sites. Sample sequencing 120 yields sequences for a plurality of nucleic acid fragments in the sample.

An analytics system performs pre-analysis processing 130. An example analytics system is described in FIG. 2B. Pre-analysis processing 130 may include, but not limited to, de-duplication of sequence reads, determining metrics relating to coverage, determining whether the sample is contaminated, removal of contaminated fragments, calling sequencing error, etc.

The analytics system performs one or more analyses 140. The analyses are statistical analyses or application of one or more trained models to predict at least a cancer status of the individual from whom the sample is derived. Different genetic features may be evaluated and considered, such as methylation of CpG sites, single nucleotide variants (SNVs), insertions or deletions (indels), copy number variation, other types of genetic variants, etc. Generally, analyses may include sample contamination detection, determining of variant characteristics of the sample, applying a panel assignment model to determine panel assignments, variant calling, featurization from called variants, and cancer classification. The cancer classifier 148 inputs the extracted features to determine a cancer prediction. The cancer prediction may be a label or a value. The label may indicate a particular cancer state, e.g., binary labels can indicate presence or absence of cancer, multiclass labels can indicate one or more cancer types from a plurality of cancer types that are screened for. The value may indicate a likelihood of a particular cancer state, e.g., a likelihood of cancer, and/or a likelihood of a particular cancer type.

The analytics system returns the prediction 150 to the healthcare provider. The healthcare provider may establish or adjust a treatment plan based on the cancer prediction. Optimization of treatment is further described in Section IV.C. Treatment. In some embodiments, the analytics system may leverage the cancer classification workflow for prognosis determination, treatment personalization, evaluation of treatment, monitoring cancer status, etc.

### I.B. Definitions

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “alternative allele” or “ALT” refers to an allele having one or more mutations relative to a reference allele, e.g., corresponding to a known gene.

As used herein, the term “alternate depth” or “AD” refers to a number of read segments in a sample that support an ALT, e.g., include mutations of the ALT.

As used herein, the term “alternate frequency” or “AF” refers to the frequency of a given ALT. The AF can be determined by dividing the corresponding AD of a sample by the depth of the sample for the given ALT.

As used herein, the term “cell free nucleic acid” or “cfNA” refers to nucleic acid fragments that circulate in an individual's body (e.g., blood) and originate from one or more healthy cells and/or from one or more unhealthy cells (e.g., cancer cells). The term “cell free DNA,” or “cfDNA” refers to deoxyribonucleic acid fragments that circulate in an individual's body (e.g., blood). Additionally, cfNAs or cfDNA in an individual's body may come from other non-human sources.

As used herein, the term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

As used herein, the term “DNA fragment,” “fragment,” or “DNA molecule” may generally refer to any deoxyribonucleic acid fragments, i.e., cfDNA, gDNA, ctDNA, etc.

As used herein, the term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

As used herein, the term “informative sequence read,” refers to a sequence read that has a called variant (e.g., copy number variant, single nucleotide variant, insertion, deletion, or other types of genetic variations).

As used herein, the term “informative score” may refer to a score for a genomic region based on informative sequence reads from a sample at the genomic region. For example, the informative score may be a count of called variants The informative score is used in context of featurization of a sample for classification.

As used herein, the term “biological sample,” “patient sample,” or “sample” refers to any sample taken from a subject, which can reflect a biological state associated with the subject, and that includes cell-free DNA. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, pericardial fluid, peritoneal fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. A biological sample can include any tissue or material derived from a living or dead subject. A biological sample can be a cell-free sample. A biological sample can comprise a nucleic acid (e.g., DNA or RNA) or a fragment thereof.

The term “nucleic acid” can refer to deoxyribonucleic acid (DNA), ribonucleic acid (RNA) or any hybrid or fragment thereof. The nucleic acid in the sample can be a cell-free nucleic acid. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “cancer” or “tumor” refers to an abnormal mass of tissue in which the growth of the mass surpasses and is not coordinated with the growth of normal tissue.

As used herein, the term “false positive” refers to a mutation incorrectly determined to be a true positive. Generally, false positives can be more likely to occur when processing sequence reads associated with greater mean noise rates or greater uncertainty in noise rates.

As used herein, the term “genomic” refers to a characteristic of the genome of an organism. Examples of genomic characteristics include, but are not limited to, those relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.).

As used herein, the term “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “indel” refers to any insertion or deletion of one or more base pairs having a length and a position (which can also be referred to as an anchor position) in a sequence read. An insertion corresponds to a positive length, while a deletion corresponds to a negative length.

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that's not cytosine; however, these are rarer occurrences. Informative cfDNA methylation can be identified as hypermethylation or hypomethylation, both of which may be indicative of cancer status.

As used interchangeably herein, the term “methylation fragment” or “nucleic acid methylation fragment” refers to a sequence of methylation states for each CpG site in a plurality of CpG sites, determined by a methylation sequencing of nucleic acids (e.g., a nucleic acid molecule and/or a nucleic acid fragment). In a methylation fragment, a location and methylation state for each CpG site in the nucleic acid fragment is determined based on the alignment of the sequence reads (e.g., obtained from sequencing of the nucleic acids) to a reference genome. A nucleic acid methylation fragment comprises a methylation state of each CpG site in a plurality of CpG sites (e.g., a methylation state vector), which specifies the location of the nucleic acid fragment in a reference genome (e.g., as specified by the position of the first CpG site in the nucleic acid fragment using a CpG index, or another similar metric) and the number of CpG sites in the nucleic acid fragment. Alignment of a sequence read to a reference genome, based on a methylation sequencing of a nucleic acid molecule, can be performed using a CpG index. As used herein, the term “CpG index” refers to a list of each CpG site in the plurality of CpG sites (e.g., CpG 1, CpG 2, CpG 3, etc.) in a reference genome, such as a human reference genome, which can be in electronic format. The CpG index further comprises a corresponding genomic location, in the corresponding reference genome, for each respective CpG site in the CpG index. Each CpG site in each respective nucleic acid methylation fragment is thus indexed to a specific location in the respective reference genome, which can be determined using the CpG index.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the terms “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 450 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the terms “sequencing” and the like as used herein refers generally to any and all biochemical processes that may be used to determine the order of biological macromolecules such as nucleic acids or proteins. Sequencing may include next generation sequencing, including whole genome sequencing, whole exome sequencing, targeted sequencing, etc. For example, sequencing data can include all or a portion of the nucleotide bases in a nucleic acid molecule such as a DNA fragment.

As used herein, the term “sequencing depth,” is interchangeably used with the term “coverage” and refers to the number of times a locus is covered by a consensus sequence read corresponding to a unique nucleic acid target molecule aligned to the locus; e.g., the sequencing depth is equal to the number of unique nucleic acid target molecules covering the locus. The locus can be as small as a nucleotide, or as large as a chromosome arm, or as large as an entire genome. Sequencing depth can be expressed as “Yx”, e.g., 50x, 100x, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a locus or a haploid genome, or a whole genome, respectively, is sequenced. When a mean depth is quoted, the actual depth for different loci included in the dataset can span over a range of values. Ultra-deep sequencing can refer to at least 100x in sequencing depth at a locus.

As used herein, the term “sequencing panel” refers to a combination of sequencing data from two or more sample (e.g., individuals).

As used herein, the term “single nucleobase variant” or “SNV” refers to a substitution of one nucleobase to a different nucleobase at a position (e.g., site) of a nucleobase sequence, e.g., a sequence read from an individual. A substitution from a first nucleobase X to a second nucleobase Y can be denoted as “X>Y.” For example, a cytosine to thymine SNV can be denoted as “C>T.”

As used herein, the term “specificity” or “true negative rate” (TNR) refers to the number of true negatives divided by the sum of the number of true negatives and false positives.

Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a woman or a child). A subject from whom a sample is taken, or is treated by any of the methods or compositions described herein can be of any age and can be an adult, infant or child.

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “true positive” (TP) refers to a subject having a condition. “True positive” can refer to a subject that has a tumor, a cancer, a pre-cancerous condition (e.g., a pre-cancerous lesion), a localized or a metastasized cancer, or a non-malignant disease. “True positive” can refer to a subject having a condition and is identified as having the condition by an assay or method of the present disclosure. As used herein, the term “true negative” (TN) refers to a subject that does not have a condition or does not have a detectable condition. True negative can refer to a subject that does not have a disease or a detectable disease, such as a tumor, a cancer, a pre-cancerous condition (e.g., a pre-cancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “white blood cell DNA,” or “wbcDNA” refers to nucleic acid including chromosomal DNA that originates from white blood cells. Generally, wbcDNA is gDNA and is assumed to be healthy DNA.

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

### I.C. Example Analytics System

FIG. 2A is an exemplary flowchart of devices for sequencing nucleic acid samples according to one or more embodiments. This illustrative flowchart includes devices such as a sequencer 220 and an analytics system 200. The sequencer 220 and the analytics system 200 may work in tandem to perform one or more steps in the processes.

In various embodiments, the sequencer 220 receives an enriched nucleic acid sample 210. As shown in FIG. 2A, the sequencer 220 can include a graphical user interface 225 that enables user interactions with particular tasks (e.g., initiate sequencing or terminate sequencing) as well as one more loading stations 230 for loading a sequencing cartridge including the enriched fragment samples and/or for loading necessary buffers for performing the sequencing assays. Therefore, once a user of the sequencer 220 has provided the necessary reagents and sequencing cartridge to the loading station 230 of the sequencer 220, the user can initiate sequencing by interacting with the graphical user interface 225 of the sequencer 220. Once initiated, the sequencer 220 performs the sequencing and outputs the sequence reads of the enriched fragments from the nucleic acid sample 210.

In some embodiments, the sequencer 220 is communicatively coupled with the analytics system 200. The analytics system 200 includes some number of computing devices used for processing the sequence reads for various applications such as variant calling or optimizing panel assignment. The sequencer 220 may provide the sequence reads in a BAM file format to the analytics system 200. The analytics system 200 can be communicatively coupled to the sequencer 220 through a wireless, wired, or a combination of wireless and wired communication technologies. Generally, the analytics system 200 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 200 may label a sequence read with one or more genes that align to the sequence read. In one embodiment, fragment length (or size) is be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome can represent the likely location within the reference genome to which the nucleic acid fragment corresponds. An output file having SAM (sequence alignment map) format or BAM (binary) format may be generated and output for further analysis.

Referring now to FIG. 2B, FIG. 2B is a block diagram of an analytics system 200 for processing DNA samples according to one embodiment. The analytics system implements one or more computing devices for use in analyzing DNA samples. The analytics system 200 includes a sequence processor 240, sequence database 245, model database 255, models 250, parameter database 265, and score engine 260. In some embodiments, the analytics system 200 performs some or all of the processes described throughout this disclosure.

Further, multiple different models 250 may be stored in the model database 255 or retrieved for use with test samples. In one example, a model is a trained cancer classifier for determining a cancer prediction for a test sample using a feature vector derived from informative fragments. The training and use of the cancer classifier will be further discussed in conjunction with Section IV. Cancer Classifier for Determining Cancer. The analytics system 200 may train the one or more models 250 and store various trained parameters in the parameter database 265. The analytics system 200 stores the models 250 along with functions in the model database 255.

During inference, the score engine 260 uses the one or more models 250 to return outputs. The score engine 260 accesses the models 250 in the model database 255 along with trained parameters from the parameter database 265. According to each model, the score engine receives an appropriate input for the model and calculates an output based on the received input, the parameters, and a function of each model relating the input and the output. In some use cases, the score engine 260 further calculates metrics correlating to a confidence in the calculated outputs from the model. In other use cases, the score engine 260 calculates other intermediary values for use in the model.

### I.D. Sample Sequencing & Processing

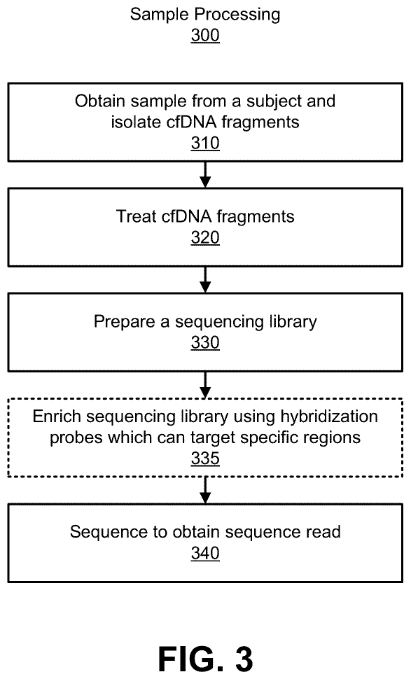

FIG. 3 is an exemplary flowchart describing a process 300 of sequencing a fragment of cfDNA to obtain a sequence read, according to one or more embodiments. An analytics system first obtains 310 a sample from an individual comprising a plurality of cfDNA molecules. In additional embodiments, the process 300 may be applied to sequence other types of DNA molecules. The process 300 is an embodiment of sample sequencing 120 of FIG. 1.

From the sample, the analytics system can isolate 310 each cfDNA molecule. The sample can be any subset of the human genome, including the whole genome. The sample can be extracted from a subject known to have, suspected of having, or not known to have cancer. The sample can include blood, plasma, serum, urine, fecal, saliva, other types of bodily fluids, or any combination thereof. In some cases, the sample can include tissue or bodily fluids extracted from tissue. In some embodiments, methods for drawing a blood sample (e.g., syringe or finger prick) can be less invasive than procedures for obtaining a tissue biopsy, which can require surgery. The extracted sample can include cfDNA and/or ctDNA. For healthy individuals, the human body can naturally clear out cfDNA and other cellular debris. If a subject has a cancer or disease, ctDNA in an extracted sample can be present at a detectable level for diagnosis.

Additionally, the extracted sample can include wbcDNA. Extracting the nucleic acid sample can further include separating the cfDNA and/or ctDNA from the wbcDNA. Extracting the wbcDNA from the cfDNA and/or ctDNA can occur when the DNA is separated from the sample. In the case of a blood sample, the wbcDNA is obtained from a buff coat fraction of the blood sample. The wbcDNA can be sheared to obtain wbcDNA fragments less than 300 base pairs in length. Separating the wbcDNA from the cfDNA and/or ctDNA allows the wbcDNA to be sequenced independently from the cfDNA and/or ctDNA. Generally the sequencing process for wbcDNA is similar to the sequencing process for cfDNA and/or ctDNA.

From the converted cfDNA molecules, a sequencing library can be prepared 330. During library preparation, unique molecular identifiers (UMI) can be added to the nucleic acid molecules (e.g., DNA molecules) through adapter ligation. The UMIs can be short nucleic acid sequences (e.g., 4-10 base pairs) that are added to ends of DNA fragments (e.g., DNA molecules fragmented by physical shearing, enzymatic digestion, and/or chemical fragmentation) during adapter ligation. UMIs can be degenerate base pairs that serve as a unique tag that can be used to identify sequence reads originating from a specific DNA fragment. During PCR amplification following adapter ligation, the UMIs can be replicated along with the attached DNA fragment. This can provide a way to identify sequence reads that came from the same original fragment in downstream analysis.

Optionally, the sequencing library may be enriched 335 for cfDNA molecules, or genomic regions, that are informative for cancer status using a plurality of targeting probes. The targeting probes are short oligonucleotides capable of hybridizing to particularly specified cfDNA molecules, or targeted regions, and enriching for those fragments or regions for subsequent sequencing and analysis. Targeting probes may be used to perform a targeted, high-depth analysis of a set of specified CpG sites of interest to the researcher. Targeting probes can be tiled across one or more target sequences at a coverage of 1X, 2X, 3X, 4X, 5X, 6X, 7X, 8X, 9X, 10X, or more than 10X. For example, targeting probes tiled at a coverage of 2X comprises overlapping probes such that each portion of the target sequence is hybridized to 2 independent probes. Targeting probes can be tiled across one or more target sequences at a coverage of less than 1X.

In one embodiment, the targeting probes are designed to enrich for DNA molecules that have been treated (e.g., using bisulfite) for conversion of unmethylated cytosines to uracils. During enrichment, targeting probes (also referred to herein as “probes”) can be used to target and pull down nucleic acid fragments informative for the presence or absence of cancer (or disease), cancer status, or a cancer classification (e.g., cancer class or tissue of origin). The probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10 s, 100 s, or 1000 s of base pairs. The probes can be designed based on a targeted sequencing panel. The probes can be designed based on a panel of targeted genes to analyze particular mutations or target regions of the genome (e.g., of the human or another organism) that are suspected to correspond to certain cancers or other types of diseases. Moreover, the probes may cover overlapping portions of a target region.

Once prepared, the sequencing library or a portion thereof can be sequenced 340 to obtain a plurality of sequence reads. The sequence reads may be in a computer-readable, digital format for processing and interpretation by computer software. The sequence reads may be aligned to a reference genome to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. A sequence read can be comprised of a read pair denoted as R1 and R2. For example, the first read R1 may be sequenced from a first end of a nucleic acid fragment whereas the second read R2 may be sequenced from the second end of the nucleic acid fragment. Therefore, nucleotide base pairs of the first read R1 and second read R2 may be aligned consistently (e.g., in opposite orientations) with nucleotide bases of the reference genome. Alignment position information derived from the read pair R1 and R2 may include a beginning position in the reference genome that corresponds to an end of a first read (e.g., R1) and an end position in the reference genome that corresponds to an end of a second read (e.g., R2). In other words, the beginning position and end position in the reference genome can represent the likely location within the reference genome to which the nucleic acid fragment corresponds. An output file having SAM (sequence alignment map) format or BAM (binary) format may be generated and output for further analysis.

One or more alternative sequencing methods can be used for obtaining sequence reads from nucleic acids in a biological sample. The one or more sequencing methods can comprise any form of sequencing that can be used to obtain a number of sequence reads measured from nucleic acids (e.g., cell-free nucleic acids), including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences,

Illumina/Solexa and Helicos Biosciences, and the sequencing-by-ligation platform from Applied Biosystems. The ION TORRENT technology from Life technologies and Nanopore sequencing can also be used to obtain sequence reads from the nucleic acids (e.g., cell-free nucleic acids) in the biological sample. Sequencing-by-synthesis and reversible terminator-based sequencing (e.g., Illumina's Genome Analyzer; Genome Analyzer II; HISEQ 2000; HISEQ 4500 (Illumina, San Diego Calif.)) can be used to obtain sequence reads from the cell-free nucleic acid obtained from a biological sample of a training subject in order to form the genotypic dataset. Millions of cell-free nucleic acid (e.g., DNA) fragments can be sequenced in parallel. In one example of this type of sequencing technology, a flow cell is used that contains an optically transparent slide with eight individual lanes on the surfaces of which are bound oligonucleotide anchors (e.g., adaptor primers). A cell-free nucleic acid sample can include a signal or tag that facilitates detection. The acquisition of sequence reads from the cell-free nucleic acid obtained from the biological sample can include obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

The one or more sequencing methods can comprise a whole-genome sequencing assay. A whole-genome sequencing assay can comprise a physical assay that generates sequence reads for a whole genome or a substantial portion of the whole genome which can be used to determine large variations such as copy number variations or copy number aberrations. Such a physical assay may employ whole-genome sequencing techniques or whole-exome sequencing techniques. A whole-genome sequencing assay can have an average sequencing depth of at least 1x, 2x, 3x, 4x, 5x, 6x, 7x, 8x, 9x, 10x, at least 20x, at least 30x, or at least 40x across the genome of the test subject. In some embodiments, the sequencing depth is about 30,000x. The one or more sequencing methods can comprise a targeted sequencing panel assay. A targeted sequencing panel assay can have an average sequencing depth of at least 50,000x, at least 55,000x, at least 60,000x, or at least 70,000x sequencing depth for the targeted panel of genes. The targeted panel of genes can comprise between 450 and 500 genes. The targeted panel of genes can comprise a range of 500±5 genes, a range of 500±10 genes, or a range of 500±25 genes.

The one or more sequencing methods can comprise paired-end sequencing. The one or more sequencing methods can generate a plurality of sequence reads. The plurality of sequence reads can have an average length ranging between 10 and 700, between 50 and 400, or between 100 and 300. The one or more sequencing methods can comprise a methylation sequencing assay. The methylation sequencing can be i) whole-genome methylation sequencing or ii) targeted DNA methylation sequencing using a plurality of nucleic acid probes. For example, the methylation sequencing is whole-genome bisulfite sequencing (e.g., WGBS). The methylation sequencing can be a targeted DNA methylation sequencing using a plurality of nucleic acid probes targeting the most informative regions of the methylome, a unique methylation database and prior prototype whole-genome and targeted sequencing assays.

### II. Panel Assignment

The analytics system assigns samples to targeted variant sequencing panels. Optimizing panel assignment is advantageous in improving sensitivity in variant calling from sequencing data obtained from the targeted variant sequencing panels, and to minimize sequencing materials and costs by close packing samples onto panels. To expand, optimizing the panel assignments can uniformize variant coverage between samples in a single panel which can improve sensitivity in variant calling. Moreover, the analytics system may group variants with similar noise rates on a single panel, to prevent missed calls among variants with high variance in noise rates. And, lastly, optimizing close packing of samples onto panels aims to minimize the number of panels needed to perform targeted sequencing for a set of samples. Minimizing the number of panels necessarily minimizes sequencing costs and materials.

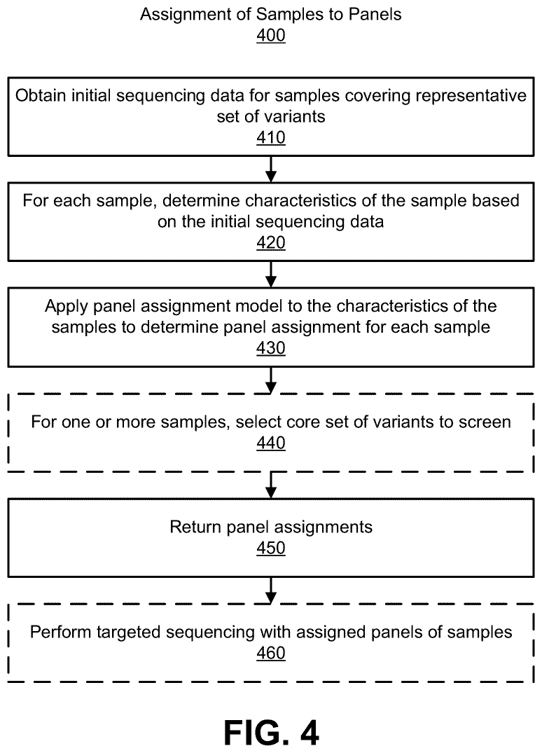

FIG. 4 is a flowchart of optimized sequencing panel assignment 400, according to one or more embodiments. The analytics system 200 may perform the method of optimized sequencing panel assignment. In other embodiments, the analytics system 200 works in conjunction with the sequencer 220. For example, the analytics system 200 may perform one or more of the analyses on the sequencing data, whereas the sequencer 220 may perform one or more of the sequencing steps or any other physical assaying steps.

The analytics system 200 obtains 410 initial sequencing data for a set of samples to be assigned to targeted variant sequencing panels. The initial sequencing data may cover a representative of variants or genomic regions, e.g., sequenced at a low sequencing depth. In other embodiments, the initial sequencing data may cover a majority or all of the genome, e.g., whole genome sequencing, or whole exome sequencing. The analytics system may determine characteristics for each variant or genomic region, e.g., a guanine and cytosine content, an error rate of targeting probes, a sequencing depth count, a presence or absence of a variant, a mean allele frequency, a total number of small variants, and an allele frequency of true variants.

The analytics system 200, for each sample, determines 420 one or more characteristics of the sample based on the initial sequencing data. As one characteristic, the analytics system may determine identify candidate variants in each sample. Relatedly, the analytics system may determine a total number of candidate variants present in the sample. The analytics system may further aggregate characteristics of each identified candidate variant under the sample. For example, a first sample has a total of 150 candidate variants, wherein each variant's characteristics are listed with the sample.

The analytics system 200 applies 430 a panel assignment model to the characteristics of the samples to determine a panel assignment for each sample. In one or more embodiments, the panel assignment model evaluates a subset of one or more characteristics. For example, the panel assignment model may assess number of variants per sample and GC content of the variants of the sample. As another example, the panel assignment model may assess error rates of the variants of the samples, e.g., in addition to the above two characteristics. The panel assignment model may determine panel assignment for each sample according to an objective function (or, alternatively, the loss function). The objective function (or, alternatively, the loss function)may be heuristically-defined to score the panel assignments based on one or more criteria. For example, in considering number of variants in each sample and GC content of the variants of the sample, the objective function scores uniformity of panel size (e.g., number of variants covered) across the panels higher and uniformity of GC content of variants within each panel higher. Alternatively, with implementing a loss function, higher variance in the panel size uniformity results in a higher loss and higher variance in GC content within each panel can result in higher loss. In one or more embodiments, the panel assignment model applies a greedy algorithm to determine panel assignment for each sample. In some embodiments, the panel assignment model may use a dynamic programming algorithm to determine the panel assignments.

Criteria can also be ranked with disparate weighting in contribution to the objective function (or, alternatively, the loss function). For example, panel uniformity is first priority, such that it receives a higher weighting to the objective function compared to GC content as second priority. The disparate weighting of criteria considered by the function can provide modularity in the panel assignment model. For example, one set of weights would prioritize some criteria in one implementation of the panel assignment model, whereas another set of weights would prioritize other criteria in a second implementation of the panel assignment model. Such modularity provides adjustable applicability of the panel assignment model to panel assignment of different sets of samples.

In additional embodiments, the panel assignment model may, upon initial assignment of samples to panels, perform a swapping operation to assess improved optimization of the panel assignments. The swapping operation may identify panel assignments negatively impacting the objective function and/or significantly contributing to the loss function as candidates for swapping. The swapping operation iteratively evaluate change to the score (objective or loss) in swapping panel assignments of samples from assigned to different panels. If the swap improves the overall optimization (maximizing the objective score and/or minimizing the loss), then the swapping operation formalizes the swap, i.e., taking the panel assignment for the first sample and assigning it to the second sample, and vice versa. The swapping operation may iteratively assess and swap samples until a stopping condition is met. One stopping condition is a maximum total number of swaps. Another stopping condition is when swap improvements have neared zero, i.e., swaps are not significantly affecting the score. A hybrid stopping condition may consider either of the maximum total swaps or when swap improvements have neared zero, e.g., when either condition is hit, then the swapping operation stops.

In one or more embodiments, the panel assignment model determines the panel assignments with one or more hard constraints. The hard constraints are inviolable constraints, i.e., the panel assignment model must satisfy the hard constraints in determining the panel assignments. For example, one hard constraint is the number of samples per panel. In another example, another hard constraint is the number of samples of one or more types per panel. In this embodiment, samples being assigned to panels may be of differing types. For example, samples being tested for tumor fraction may be deemed “test samples,” whereas samples of known tumor fractions (i.e., titrated at precise tumor fractions from tissue biopsy samples) may be deemed “benchmarking samples.” One hard constraint may limit the number of benchmarking samples that may be assigned to any given panel, e.g., at most 1 per panel. Another hard constraint may involve placing each benchmarking sample on some number of panels, e.g., on exactly 3 panels.

In some embodiments, the analytics system 200, for one or more samples, selects 440 a core set of variants to screen. In some embodiments, one or more of the samples may have greater than a threshold number of variants to be screened by the targeted sequencing panel. For example, the targeted sequencing panels are capped at evaluating up to 500 variants for any given sample. For a sample with 600 variants, the analytics system 200 identifies a subset of the 600 variants to screen for on the assigned panel. To identify the subset, the analytics system 200 may apply a selection algorithm. The selection algorithm may leverage a function to score the variants of the sample based on characteristics of the variants. In one or more embodiments, the function may be similar to the objective function and/or the loss function of the panel assignment model. The function may assess GC content of a variant, error rate of an associated probe, a position in the genome of the variant, sequencing depth of the variant, presence or absence of variant, mean allele frequency, total number of small variants, allele frequency of true variants, or some combination thereof. The selection algorithm may seek to optimize selected variants with the context of other samples on the panel. For example, if one variant has high frequency as present on a large percentage of samples of a panel, that one variant may be weighted higher as having a sequencing-cost-saving advantage. Such advantage may be balanced against other factors, e.g., sensitivity of the variant calling.

The analytics system 200 returns 450 the panel assignment for each sample in the set of samples. Each panel includes variants to be screened for and includes the assigned samples. The panel may further include probes targeting the identified variants.

The sequencer 220 may perform 460 the targeted sequencing with the assigned panels. For example, the targeted sequencing is further described in Section I.D. Sample Sequencing and Processing.

### III. Variant Calling

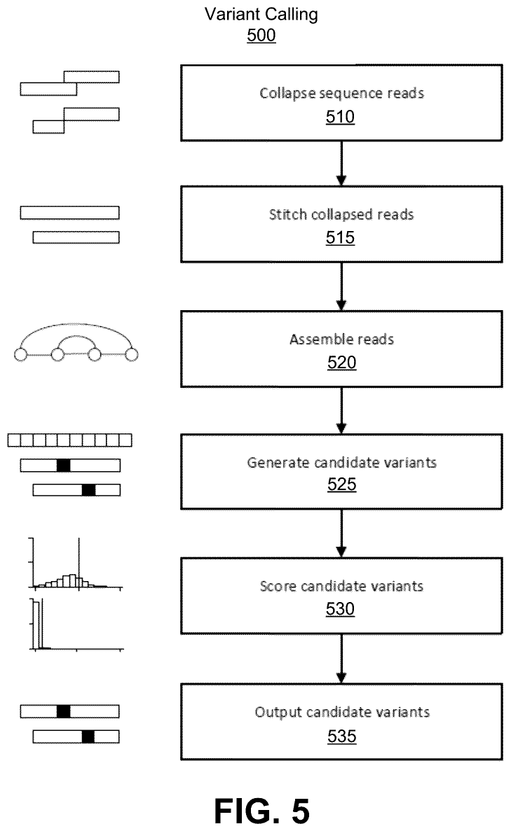

The analytics system can call variants from sequencing data for a particular sample. In particular, FIG. 5 is a flowchart of a workflow for determining variants of sequence reads according to one embodiment. The sequencing data may be obtained from targeted sequencing with panels generated by the panel assignment model, e.g., as described in Section II. Panel Assignment.

In some embodiments, the analytics system 200 uses the workflow 500 to perform variant calling (e.g., for SNVs and/or indels) based on input sequencing data. Further, the analytics system 200 can obtain the input sequencing data from an output file associated with nucleic acid sample prepared using the workflow 100 described above. The workflow 500 includes, but is not limited to, the following steps, which are described with respect to the components of the analytics system 200. In other embodiments, one or more steps of the workflow 500 can be replaced by a step of a different process for generating variant calls, e.g., using Variant Call Format (VCF), such as HaplotypeCaller, VarScan, Strelka, or SomaticSniper.

At step 510, the analytics system 200 collapses aligned sequence reads of the input sequencing data. In one embodiment, collapsing sequence reads includes using UMIs, and optionally alignment position information from sequencing data of an output file (e.g., from the workflow 100 shown in FIG. 1) to collapse multiple sequence reads into a consensus sequence for determining the most likely sequence of a nucleic acid fragment or a portion thereof. Since the UMIs are replicated with the ligated nucleic acid fragments through enrichment and PCR, the sequence processor can determine that certain sequence reads originated from the same molecule in a nucleic acid sample. In some embodiments, sequence reads that have the same or similar alignment position information (e.g., beginning and end positions within a threshold offset) and include a common UMI are collapsed, and the sequence processor generates a collapsed read (also referred to herein as a consensus read) to represent the nucleic acid fragment. The sequence processor designates a consensus read as “duplex” if the corresponding pair of collapsed reads have a common UMI, which indicates that both positive and negative strands of the originating nucleic acid molecule is captured; otherwise, the collapsed read is designated “non-duplex.” In some embodiments, the sequence processor can perform other types of error correction on sequence reads as an alternate to, or in addition to, collapsing sequence reads.

At step 515, the analytics system 200 stitches the collapsed reads based on the corresponding alignment position information. In some embodiments, the sequence processor compares alignment position information between a first read and a second read to determine whether nucleobase base pairs of the first and second reads overlap in the reference genome. In one use case, responsive to determining that an overlap (e.g., of a given number of nucleobase bases) between the first and second reads is greater than a threshold length (e.g., threshold number of nucleobase bases), the sequence processor designates the first and second reads as “stitched”; otherwise, the collapsed reads are designated “unstitched.” In some embodiments, a first and second read are stitched if the overlap is greater than the threshold length and if the overlap is not a sliding overlap. For example, a sliding overlap can include a homopolymer run (e.g., a single repeating nucleobase base), a dinucleobase run (e.g., two-nucleobase base sequence), or a trinucleobase run (e.g., three-nucleobase base sequence), where the homopolymer run, dinucleobase run, or trinucleobase run has at least a threshold length of base pairs.

At step 520, the analytics system 200 assembles reads into paths. In some embodiments, the analytics system 200 assembles reads to generate a directed graph, for example, a de Bruijn graph, for a target region (e.g., a gene). Unidirectional edges of the directed graph represent sequences of k nucleobase bases (also referred to herein as “k-mers”) in the target region, and the edges are connected by vertices (or nodes). The sequence processor aligns collapsed reads to a directed graph such that any of the collapsed reads can be represented in order by a subset of the edges and corresponding vertices.

In some embodiments, the analytics system 200 determines sets of parameters describing directed graphs and processes directed graphs. Additionally, the set of parameters can include a count of successfully aligned k-mers from collapsed reads to a k-mer represented by a node or edge in the directed graph. The analytics system 200 stores, e.g., in the sequence database 245, directed graphs and corresponding sets of parameters, which can be retrieved to update graphs or generate new graphs. For instance, the analytics system 200 can generate a compressed version of a directed graph (e.g., or modify an existing graph) based on the set of parameters. In one use case, in order to filter out data of a directed graph having lower levels of importance, the sequence processor removes (e.g., “trims” or “prunes”) nodes or edges having a count less than a threshold value, and maintains nodes or edges having counts greater than or equal to the threshold value.

In one embodiments, the analytics system 200 can store sequencing data in the sequence database 245 (e.g., variants and normals), which can be used to detect presence, absence, or level of a feature values (e.g., GC content, an error rate, a sequencing depth count, a presence or absence of a variant, a mean allele frequency, a total number of small variants, and an allele frequency of true variants) in a sample from a subject, and/or otherwise predict cost associated with the variant (e.g., per-site cost values and relative costs). The sequence database 245 can also store sequencing data processed by the analytics system 200, but can also store sequencing data not processed by the analytics system 200, such as sequencing data uploaded from an external source and/or otherwise retrieved from external or publicly available databases.

At step 525, the analytics system 200 generates candidate variants from the paths assembled by the sequence processor 240. In one embodiment, the analytics system 200 generates the candidate variants by comparing a directed graph (which can have been compressed by pruning edges or nodes in step 510) to a reference sequence of a target region of a genome. The analytics system 200 can align edges of the directed graph to the reference sequence, and records the genomic positions of mismatched edges and mismatched nucleobase bases adjacent to the edges as the locations of candidate variants. Additionally, the analytics system 200 can generate candidate variants based on the sequencing depth of a target region. In particular, the analytics system 200 can be more confident in identifying variants in target regions that have greater sequencing depth, for example, because a greater number of sequence reads help to resolve (e.g., using redundancies) mismatches or other base pair variations between sequences.

In one embodiment, the analytics system 200 generate candidate variants using a variant model to determine expected noise rates for sequence reads from a subject. The variant model can be a Bayesian hierarchical model, though in some embodiments, the analytics system 200 uses one or more different types of models. Moreover, a Bayesian hierarchical model can be one of many possible model architectures that can be used to generate candidate variants and which are related to each other in that they all model position-specific noise information in order to improve the sensitivity/specificity of variant calling. More specifically, the analytics system 200 may train the variant model using samples from healthy individuals to model the expected noise rates per position of sequence reads.

Further, multiple different models can be stored in the model database 255 or retrieved for application post-training. For example, a first model is trained to model SNV noise rates and a second model is trained to model indel noise rates. Further, the score engine 260 can use parameters of the variant model to determine a likelihood of one or more true positives in a sequence read. The score engine 260 can determine a quality score (e.g., on a logarithmic scale) based on the likelihood. For example, the quality score is a Phred quality score:

$$Q = - 10 \cdot {log}_{10} P$$

where P is the likelihood of an incorrect candidate variant call (e.g., a false positive).

At step 530, the score engine 260 scores the candidate variants based on the variant model or corresponding likelihoods of true positives or quality scores.

At step 535, the analytics system 200 outputs the candidate variants. In some embodiments, the analytics system 200 outputs some or all of the determined candidate variants along with the corresponding scores. Downstream systems, e.g., external to the analytics system 200 or other components of the analytics system 200, can use the candidate variants and scores for various applications including, but not limited to, predicting presence of cancer, disease, or germline mutations.

In one embodiment, candidate variants are outputted for both cfDNA and/or ctDNA and wbcDNA. Herein, generally, candidate variants for wbcDNA are “normals” while candidate variants for cfDNA and/or ctDNA are “variants.” Various detection methods and models can compare variants to normals to determine if the variants include signatures of cancer or any other disease. In various embodiments, normals and variants can be generated using any other process, any number of samples (e.g., a tumor biopsy or blood sample), or accessed from a database storing candidate variants.

In one embodiment, the outputted candidate variants are used in the methods described herein to generate an optimized sequencing panel assignment.

Further details regarding variant calling can be found in U.S. application Ser. No. 16/119,961 entitled “Identifying False Positive Variants Using a Significance Model,” filed on Aug. 31, 2018, which is incorporated by reference herein. Further details regarding identifying copy number aberrations or copy number variants can be found in U.S. application Ser. No. 15/853,314 entitled “Base Coverage Normalization and Use Thereof in Detecting Copy Number Variation,” filed on Dec. 22, 2017, and U.S. application Ser. No. 16/352,214 entitled “Identifying Copy Number Aberrations,” filed on Mar. 13, 2019, both of which are incorporated by reference herein.

### IV. Cancer Classifier

Cancer classification involves extracting genetic features and applying one or more models to the extracted features to determine a cancer prediction. The analytics system aggregates extracted features into a feature vector which can then be input into a trained cancer prediction model to determine a cancer prediction based on the input feature vector. The cancer prediction may comprise one or more labels and/or one or more values. One label may be binary, indicating a presence or absence of cancer in the test subject. Another label may be multiclass, indicating one or more particular cancer types from a plurality of screened cancer types. One value may indicate a likelihood of presence of cancer. Another value may indicate a likelihood of absence of cancer. Yet another value may otherwise indicate another prognosis of the cancer. For example, the value may quantify a progression and/or an aggression of the cancer. Still yet, one embodiment of the cancer classifier may output a predicted tumor fraction in the sample. Such cancer prediction may be useful in monitoring progression of cancer, evaluating efficacy in treatment, detecting cancer recurrence, measuring minimal residual disease, etc.

In some embodiments, a cancer classifier may be a machine-learned model comprising a plurality of classification parameters and a function representing a relation between the feature vector as input and the cancer prediction as output. Inputting the feature vector into the function with the classification parameters yields the cancer prediction. The machine-learned model may be trained using training samples derived from individuals with known cancer diagnoses. The training samples may be divided into cohorts of varying labels. For example, there may be a cohort of training samples for each cancer type. As another example, training samples may have known tumor fraction through titration of tissue biopsy samples (also referred to as “benchmarking samples”).

### IV.A. Training of Cancer Classifier

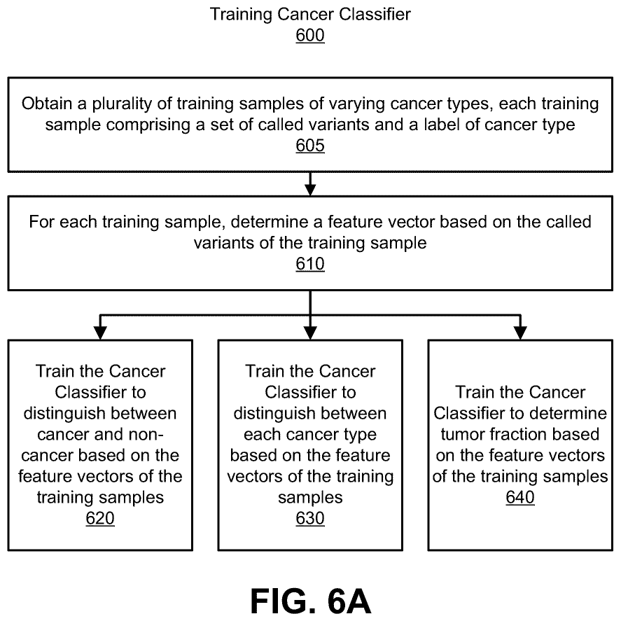

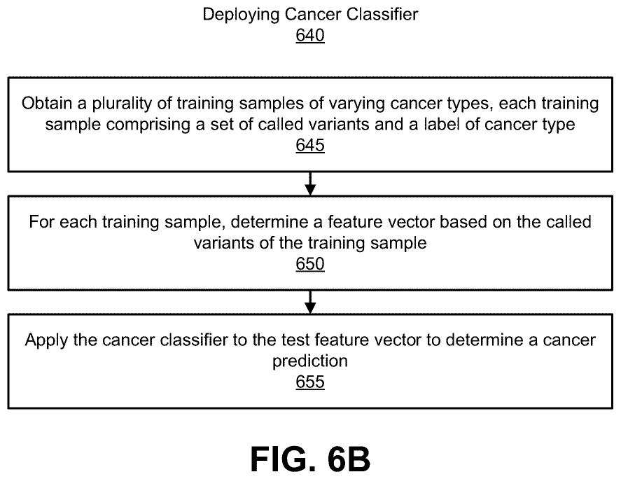

FIG. 6A is a flowchart describing a process 600 of training a cancer classifier, according to an embodiment. The analytics system obtains 610 a plurality of training samples each training sample comprising a set of called variants and a label of a cancer type. The plurality of training samples can include any combination of samples from healthy individuals with a general label of “non-cancer,” samples from subjects with a general label of “cancer” or a specific label (e.g., “breast cancer,” “lung cancer,” etc.). The label may also indicate a tumor fraction, e.g., represented as a percentage. The training samples from subjects for one cancer type may be termed a cohort for that cancer type or a cancer type cohort.

The analytics system determines 620, for each training sample, a feature vector based on the called variants of the training sample. The feature may be binary, indicating presence or absence of the variant. The feature may indicate a percentage of sequence reads in a sample's sequencing data that indicate presence or another feature may indicate the percentage indicating absence of the variant. In some embodiments, the feature may relate to the allele frequency of the variant. In some embodiments, the feature may aggregate count or percentage of sequence reads including called variants in partitioned genomic regions. Other types of features based on the called variants may be implemented.

Once all features are determined for a training sample, the analytics system can determine the feature vector as a vector of elements including, for each element, one of the features. The analytics system can normalize the features, e.g., based on sequencing depth, or a coverage of each variant.

Additional approaches to featurization of a sample can be found in: U.S. application Ser. No. 16/384, 784 entitled “Multi-Assay Prediction Model for Cancer Detection, U.S. application Ser. No. 16/579,805 entitled “Mixture Model for Targeted Sequencing;” which are incorporated by reference in their entirety.

With the feature vectors of the training samples, the analytics system may train the cancer classifier in any of a number of ways. In one embodiment, the analytics system trains 620 a binary cancer classifier to distinguish between cancer and non-cancer based on the feature vectors of the training samples. In this manner, the analytics system uses training samples that include both non-cancer samples from healthy individuals and cancer samples from subjects. Each training sample can have one of the two labels “cancer” or “non-cancer.” In this embodiment, the classifier outputs a cancer prediction indicating the likelihood of the presence or absence of cancer.

In another embodiment, the analytics system trains 630 a multiclass cancer classifier to distinguish between many cancer types (also referred to as tissue of origin (TOO) labels). Cancer types can include one or more cancers and may include a non-cancer type (may also include any additional other diseases or genetic disorders, etc.). To do so, the analytics system can use the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood.

In a third embodiment, the analytics system trains 640 the cancer classifier to determine a tumor fraction based on the feature vectors of the training samples. In such an embodiment, the tumor fraction indicates an amount of tumor signal in the sample, e.g., which may serve as a proxy indicator for cancer progression. To train such a classifier, the analytics system may train the classifier in a supervised manner to input the feature vectors of the training samples and to predict the known tumor fractions of the training samples. Such training samples with known tumor fractions may be referred to as benchmarking samples. The cancer classifier may be trained as a machine-learning regression model.

In the various embodiments, the analytics system trains the cancer classifier by inputting sets of training samples with their feature vectors into the cancer classifier and adjusting classification parameters so that a function of the classifier accurately relates the training feature vectors to their corresponding label (e.g., cancer status, cancer type, or tumor fraction). The analytics system may group the training samples into sets of one or more training samples for iterative batch training of the cancer classifier. After inputting all sets of training samples including their training feature vectors and adjusting the classification parameters, the cancer classifier can be sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

The classifier can include a logistic regression algorithm, a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

### IV.B. Deployment of Cancer Classifier

During use of the cancer classifier, the analytics system can obtain a test sample from a subject of unknown cancer type. The analytics system may process the test sample comprised of DNA molecules to call variants in the test sample's genetic data. The analytics system can determine a test feature vector for use by the cancer classifier according to similar principles discussed in the process 600. The analytics system can generate a feature vector for the test sample to be input into the cancer classifier. For example, the cancer classifier receives as input feature vectors information relating to 2,000 or so variants in the human genome. The analytics system can thus determine a test feature vector inclusive of features for the 2,000 variants based on the called variants for the test sample.

The analytics system can then input the test feature vector into the cancer classifier. The function of the cancer classifier can then generate a cancer prediction based on the classification parameters trained in the process 600 and the test feature vector. In the first manner, the cancer prediction can be binary and selected from a group consisting of “cancer” or non-cancer.” In the second manner, the cancer prediction is selected from a group of many cancer types and “non-cancer.” In the third manner, the cancer prediction indicates a tumor fraction present in the sample. In additional embodiments, the cancer prediction has predictions values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains cancer classifiers together. For example, the analytics system can input the test feature vector into the cancer classifier trained as a binary classifier in step 620 of the process 600. The analytics system can receive an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types, e.g., as described in step 630 of the process 600. The multiclass cancer classifier can receive the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%. In other embodiments, the analytics system may further input the test feature vector into a cancer classifier trained to estimate tumor fraction, e.g., as described in step 640 of the process 600. For example, the estimated tumor fraction may be represented as a percentage, e.g., 25% tumor fraction. When chained with the multiclass cancer classifier, the estimated tumor fraction may designate differing fractions for each cancer type.

According to generalized embodiment of binary cancer classification, the analytics system can determine a cancer score for a test sample based on the test sample's sequencing data (e.g., methylation sequencing data, small variant sequencing data, other DNA sequencing data, RNA sequencing data, etc.). The analytics system can compare the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

The classifier may be used to determine the disease state of a test subject, e.g., a subject whose disease status is unknown. The method can include obtaining a test genomic data construct (e.g., single time point test data), in electronic form, that includes a value for each genomic characteristic in the plurality of genomic characteristics of a corresponding plurality of nucleic acid fragments in a biological sample obtained from a test subject. The method can then include applying the test genomic data construct to the test classifier to thereby determine the state of the disease condition in the test subject. The test subject may not be previously diagnosed with the disease condition.

The classifier can be a temporal classifier that uses at least (i) a first test genomic data construct generated from a first biological sample acquired from a test subject at a first point in time, and (ii) a second test genomic data construct generated from a second biological sample acquired from a test subject at a second point in time.

The trained classifier can be used to determine the disease state of a test subject, e.g., a subject whose disease status is unknown. In this case, the method can include obtaining a test time-series data set, in electronic form, for a test subject, where the test time-series data set includes, for each respective time point in a plurality of time points, a corresponding test genotypic data construct including values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points. The method can then include applying the test genotypic data construct to the test classifier to thereby determine the state of the disease condition in the test subject. The test subject may not be previously diagnosed with the disease condition.

### V. Applications

In some embodiments, the methods, analytics systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment. In additional embodiments, the methods, analytics system, and/or classifiers can be implemented to detect contamination sources in the sample processing and analysis workflow. Upon detection of contamination and/or contamination sources, the analytics system can aid in performing remedial measures to mitigate the contamination and the negative effects thereof (e.g., skewing results, biasing training of the classifiers, etc.).

### V.A. Panel, Assignment Generation

In one example embodiment, the analytics system generates a sequencing panel assignment with the aim of reducing cost of sequencing without compromising Limit of Detection (LoD).

The analytics system 200 obtains sequencing data (e.g., test sequences) for a set of samples (e.g., here samples that meet a set of criteria described herein). The first sequencing data can be the CCGA indicator set but could be another set of genomic regions to be analyzed. The sequencing data is associated with a number of test sequences, and is associated with feature values (e.g., a GC content, an error rate, a sequencing depth count, a presence or absence of a variant, a mean allele frequency, a total number of small variants, and an allele frequency of true variants).

The analytics system 200 selects a feature value to be analyzed for each of the samples. For example, the feature value can be a GC content, an error rate, a sequencing depth count, a presence or absence of a variant, a mean allele frequency, a total number of small variants, and an allele frequency of true variants in the sequencing data. Other feature values are also possible.

The analytics system 200 ranks the samples according to their feature values at the set of genomic regions. For example, the sample with the highest feature value is ranked first, while the sample with the lowest feature value is ranked last.

After the samples are ranked based on decreasing feature value, the method includes applying a greedy algorithm to add a next-highest ranked sample of the remaining ranked samples to a panel, wherein the panel to which the sample is sorted comprises the lowest value of feature values.

In some embodiments, the analytics system 200 can access one or more additional sets of feature values and apply the machine learning model to the samples based on the additional set of set of feature values. In doing so, the analytics system 200 can identify one or more additional subsets of feature values for consideration when applying the greedy algorithm to assignment of samples to panels.

The analytics system 200 generates the optimized sequencing panel assignment by applying a seed and swap approach to compiling a panel. In some embodiments, the analytics system 200 iterates through samples to assign the samples to a panel, determines mean of feature values for each panel, swapping two samples between two different panels, and measuring deviation of mean feature value for each of the two different panels following the swap. The sequencing panel generator includes repeating these steps for a pre-specified number of swaps, thereby generating a panel assignment based on the feature values from the sequencing data. In such cases. the repeating step is performed until the reduction in the mean feature value is below a threshold.

There are several filtering methods that can improve sequencing panel assignments. In a first example, the sequencing panel generator can only derive feature values for genomic regions having variants in a threshold number of sequences in the sequencing data. In a second example, the sequencing panel generator can duplicate, or remove duplications, of a genomic region from a panel to increase detection capability. In a third example, a system administrator can remove genomic regions from the analysis. In a fourth example, a system administrator can remove samples from the sequencing panel. Finally, the sequencing panel generator can remove feature values from the panel based on a feature value blacklist. The feature value blacklist can include patented feature values, feature values known to cause false positives, or any other feature value that could decrease the detection capability of a panel.

### V.A.I Example Panel Assignment Considerations

In As described herein, the analytics system 200 generates a sequencing panel assignment with the aim of reducing cost of sequencing without compromising Limit of Detection (LoD).

As noted above, the aim of the methods described herein is to reduce the cost of sequencing without compromising the Limit of Detection (LoD). In determining the steps needed for sequencing panel optimization, a consideration is the type of samples (e.g., target and control samples used to develop the methods). Here, target samples selected for testing included one or more of (i) to be undetected by past classifiers (i.e., detectability), (ii) have available plasma tubes (i.e., available plasma), and (iii) contain current tumor fraction estimates less than 1%, if available (i.e., low estimated TF). As noted above, the pool of potential samples included samples from the Circulating Cell Genome Atlas 1 Study (CCGA1) and the Circulating Cell Genome Atlas 2 Study (CCGA2).

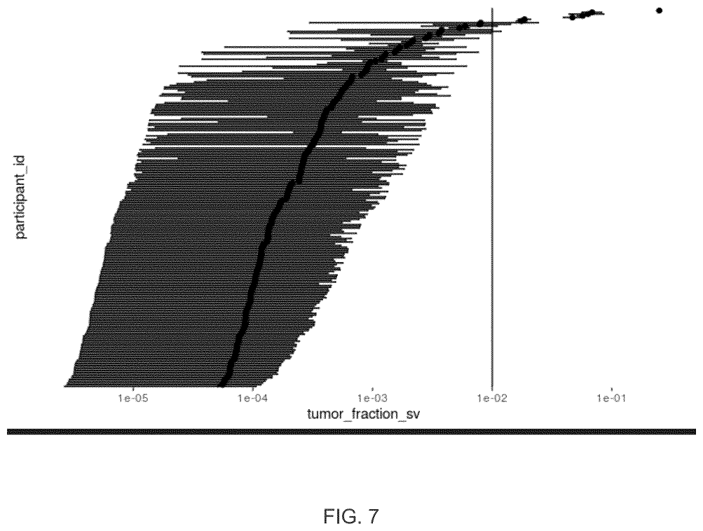

Applying the (i) “detectability” filter to the CCGA1 and CCGA2 samples revealed about 297 participants. For example, clinically evaluable participants with data for variant calling already available were analyzed. In particular, participants with WGBS of tumor biopsy and WGS of cfDNA were selected from Circulating Cell Genome Atlas 1 Study (CCGA1) and Circulating Cell Genome Atlas 2 Study (CCGA2). For samples from CCGA1, a threshold Mscore below 98% spec score cutoff (0.755) revealed 129 patients. For samples form CCGA2, participants that were undetected across all samples (v0.5 tube1/tube2, v2 lanes 12/34) at 99.4% spec score cutoff revealed 196 participants. Of the 297 participants that met the detectability criteria, only 236 have enough available plasma for further analysis. Of the 236 participants that met the first two criteria (i.e., not previously detected and having available plasma tubes) 210 participants had a Tumor Fraction estimate <1% (see FIG. 7). Seven participants had a TF estimate>1% and 19 participants had no TF estimate, so each of these 26 participants were excluded. These 210 participants were used to test the methods described herein.

Benchmarking samples are contrived titrations with known tumor fractions (e.g., any of the tumor fraction values described herein). In some embodiments, the estimated tumor fraction (TF) of a benchmarking sample is at least 10%. In one or more embodiments, benchmarking samples can be used for benchmarking of variant calling. In one embodiment, benchmarking samples can also be used for TF estimation.

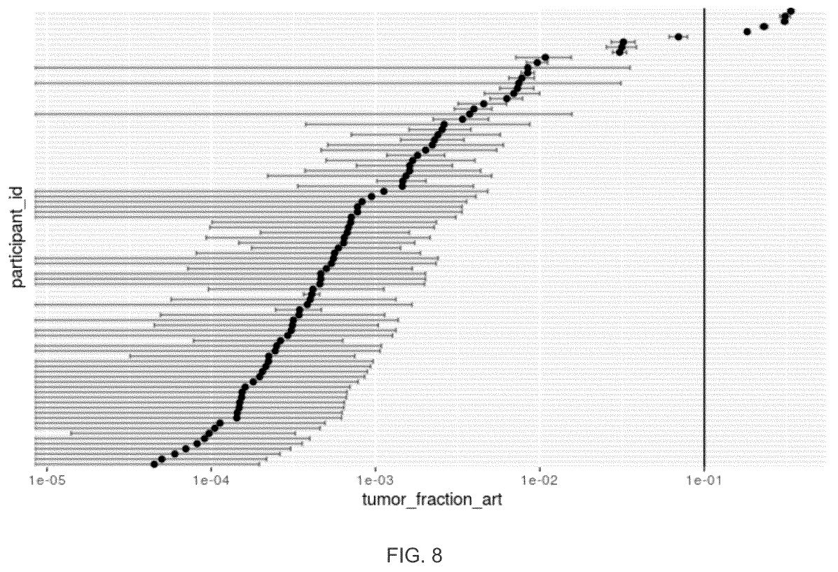

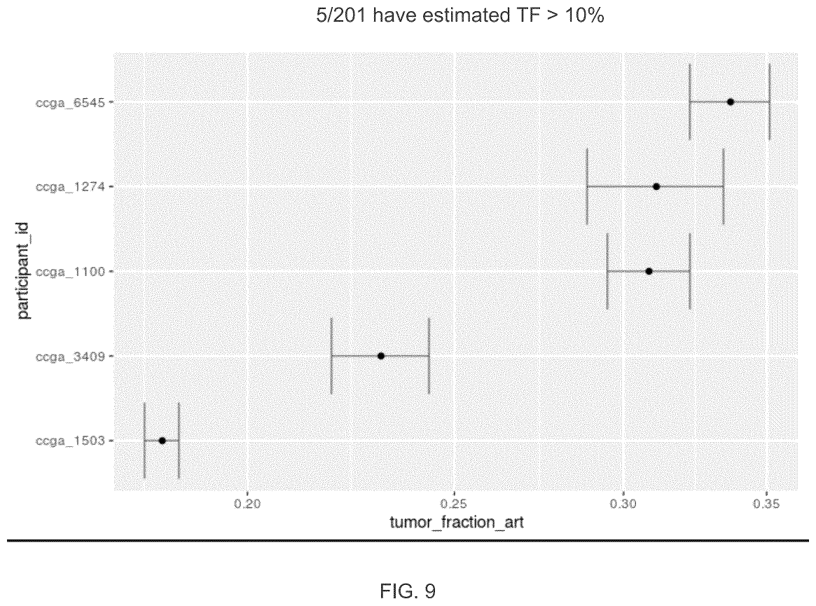

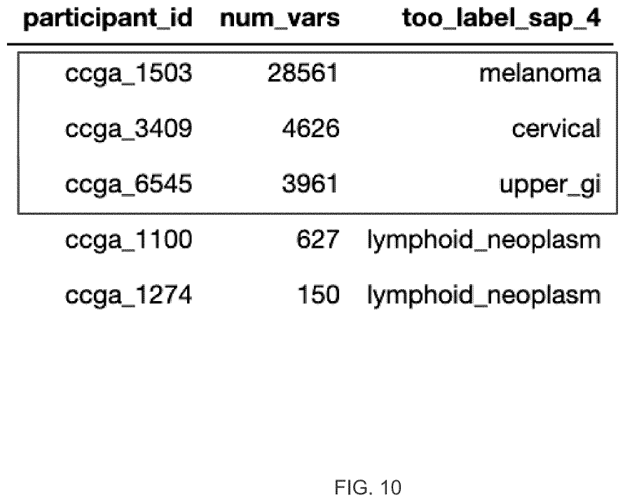

Benchmarking samples were selected from CCGA1 participants that included WBC WGS data. Of the 201 participants whose samples included the requisite amount of material for analysis, only five of the 201 had estimated tumor fraction greater than 10% (See FIG. 8 and FIG. 9). From these five samples, the three samples with the most variant calls were selected as Benchmarking samples (See FIG. 10).

One major consideration when developing methods for improving sequencing panel assignments is to find but avoid going below the lowest tumor fraction (e.g., limit of detection) that can be detected using a feature value (e.g., SNPs/variants) for a set of genomic regions.

To determine the lowest tumor fraction (e.g., limit of detection) for each participant, simulations were run including the expected number of total fragments and alt-allele containing fragments under a range of possible tumor fractions as well as pure noise (i.e., 0% TF). The limit of detection (LoD) refers to the lowest TF with good separation of the alt fraction distribution from noise. For the LoD, the main parameters were: number of collapsed fragments per target and error rates of collapsed fragments.

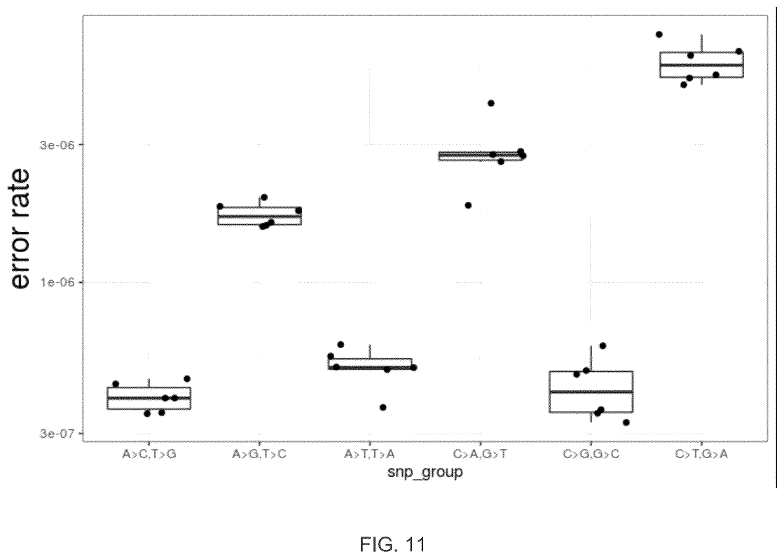

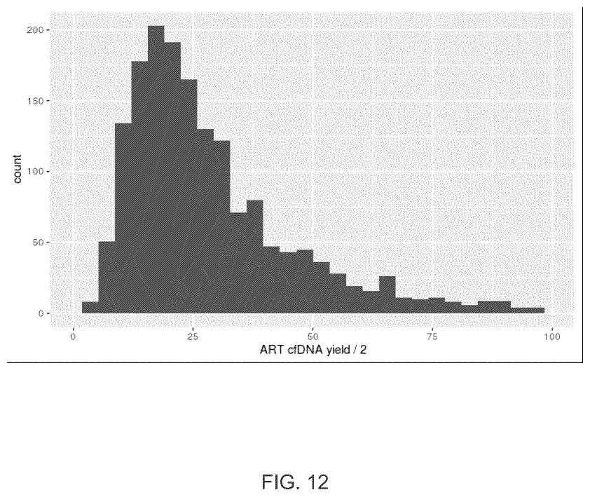

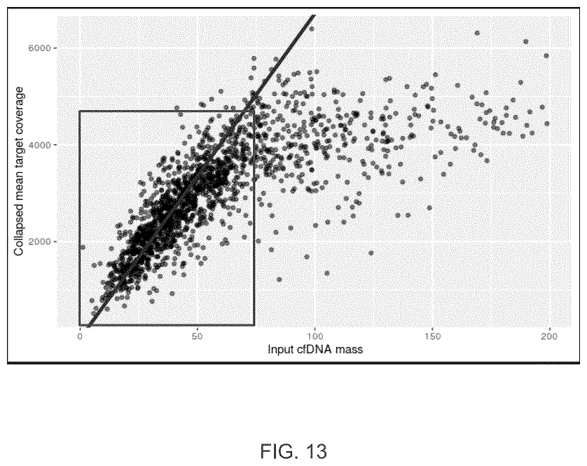

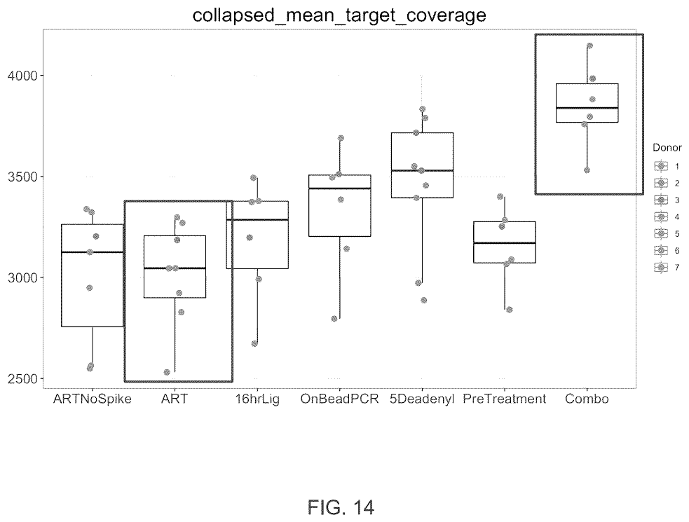

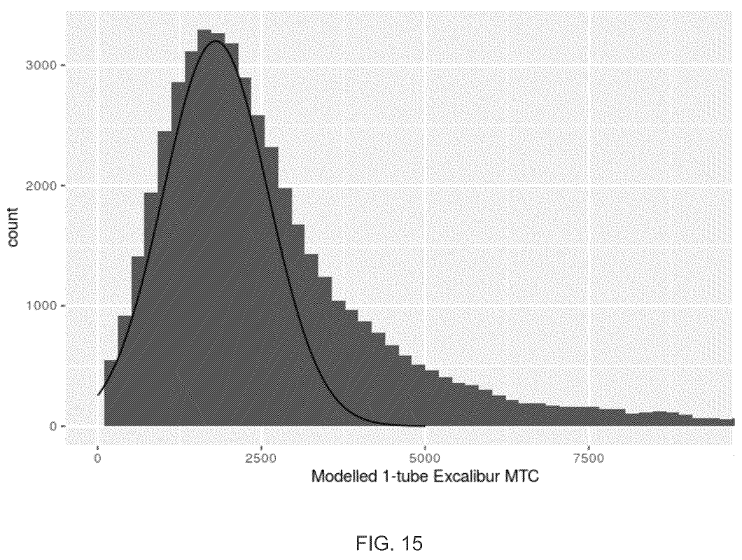

As shown in FIG. 11, the error rate for the different conversion types reveals that when using variants as the feature value, variant selection will need to account for conversion type. Additionally, FIGS. 12-14 show that the amount of cfDNA is an additional consideration when considering LoD.

In one embodiment, variants are used as the feature value. For example, for each patient, sequencing data including variant calls that pass threshold were analyzed. Variant calls were selected, for example up to N variant calls, and prioritized based on noise rate and allele fraction in the tumor fraction. For each tumor fraction (TF) a simulation was performed in logspace (−7, −3, and 50): alt_rates=Tumor Fraction*allele_fractions+(1-TF)*noise_rates. About 10,000 simulations were run: (1) sample total collapsed target coverage (same across all sites); (2) sample alt counts for each variant according to alt_rates and total coverage; and (3) alt frac=sum (alt counts)/sum (total counts). To ensure good separation of alt fraction from noise distribution a noise cutoff was used: noise_cutoff_quantile (noise_alt_fracs_0.99). The

LoD for tumor fractions were identified as those with TF with 95% of alt_frac samples>noise cutoff.

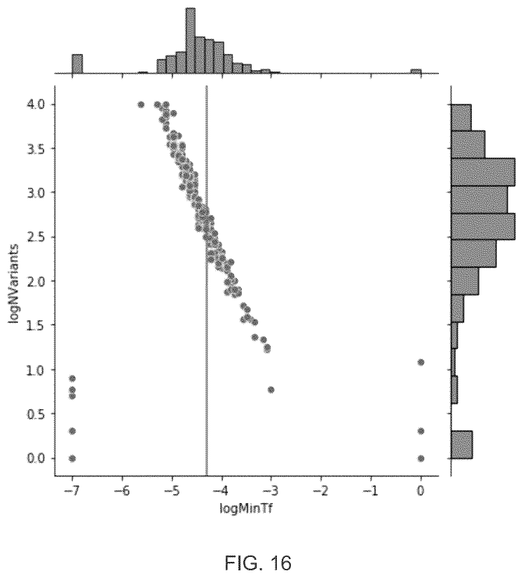

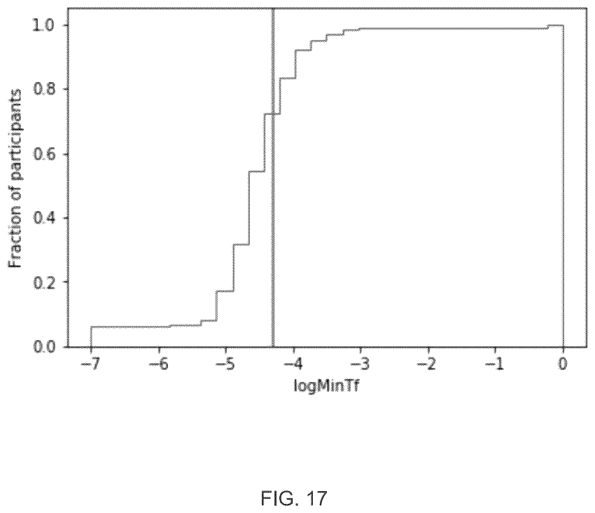

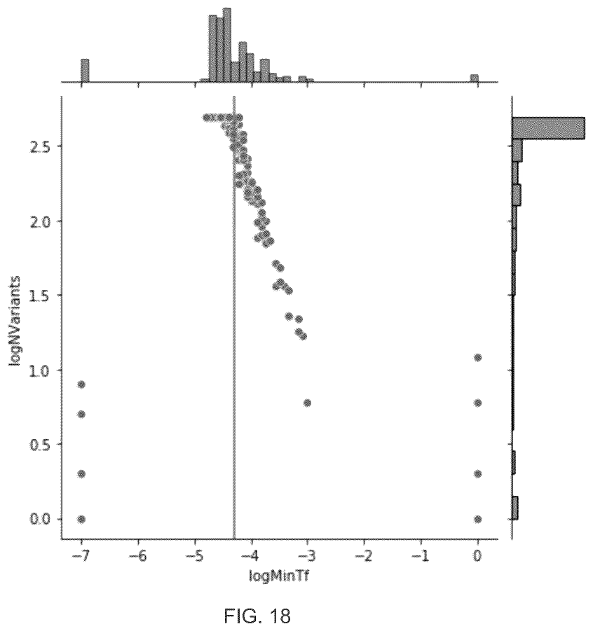

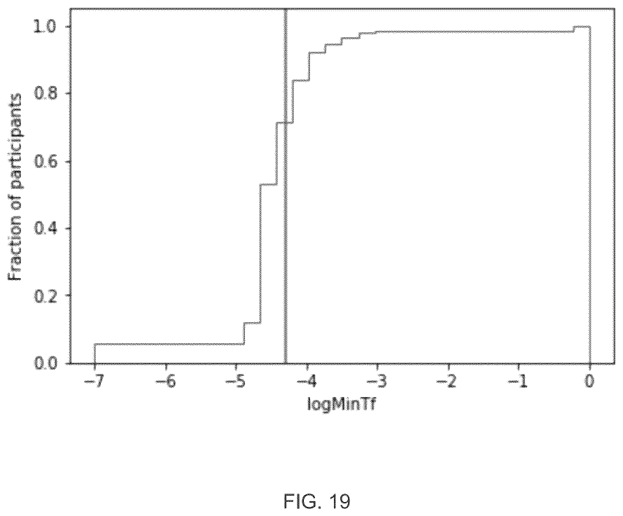

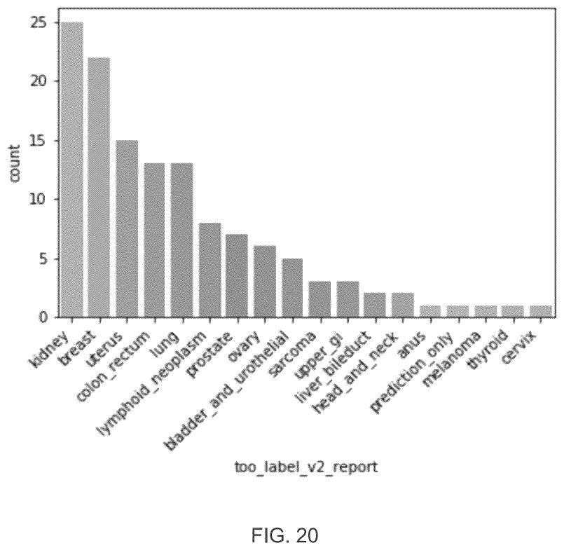

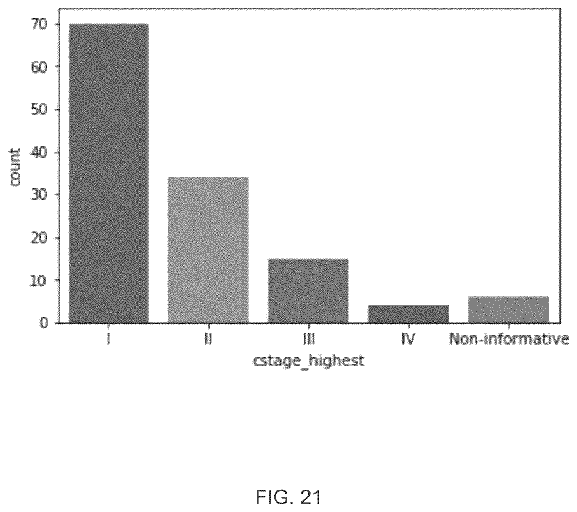

Results of LoD modelling for samples (patients) and SNPs as the feature value are shown in FIG. 16 and FIG. 17. The expected LoD was largely driven by the number of variants. Here about 300 to about 400 variants were needed to get below 5 E-5. FIG. 18 and FIG. 19 show simulations when the subset of variants (i.e., the subset of genomic regions pertaining to those variants) is set to a max of 500 variants per participant. 129/201 target participants have expected LoD<5E-5 (excluding samples with number of variants less than 20). The LoD<5E-5 was largely driven by number of variants. An inflection point of TF LoD is around the target of LoD. Analysis of the 129 target participants having an expected LoD<5E-5 revealed specific cancer types (FIG. 20) and stages (FIG. 21).

The number of variants per participant can be determined empirically. In some embodiments, determining the number of variants per participant includes (i) confidence of the variant call, (ii) error rate of the site, and (iii) ease of sequencing/availability of cfDNA. In one embodiment, the number of variants per participant is less than 500 variants.

In one embodiment, determining (i) confidence of the variant call includes a log-likelihood of a true call versus noise. This can be represented as:

$$L L R = log ( \frac{Binom ( alt , tot , 0.5 )}{Binom ( alt , tot , noise )}$$

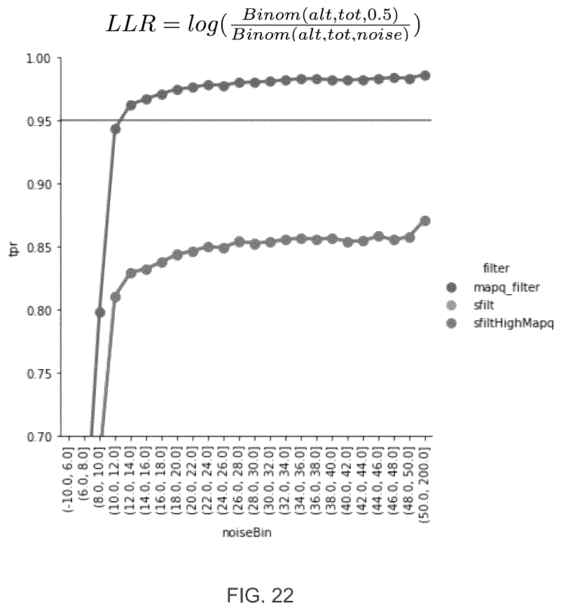

As shown in FIG. 22, little change in PPV is seen beyond 16. In one embodiment, PPV cutoff is 25 (see FIG. 22).

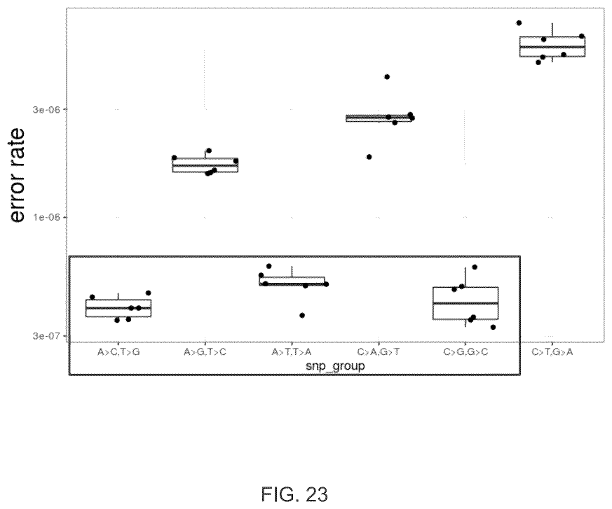

In one embodiment, determining (ii) “error of the site” (i.e., site of the variant) depends, at least in part, on the conversion type of the variant (e.g., SNP). FIG. 23 shows error rates for different conversion types. This shows greater error rates for A>G, T>C; C>A, G>T; and C>T, G>A. The box in FIG. 23 highlights the conversion types having the lowest error rates, including: A>C, T>G; A>T, T>A; and C>G, G>C. The variants having the lowest error rates met the criteria for “error rate of the site.”

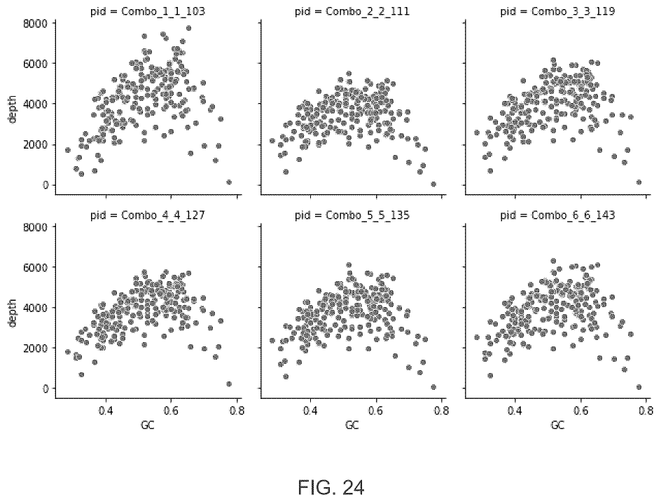

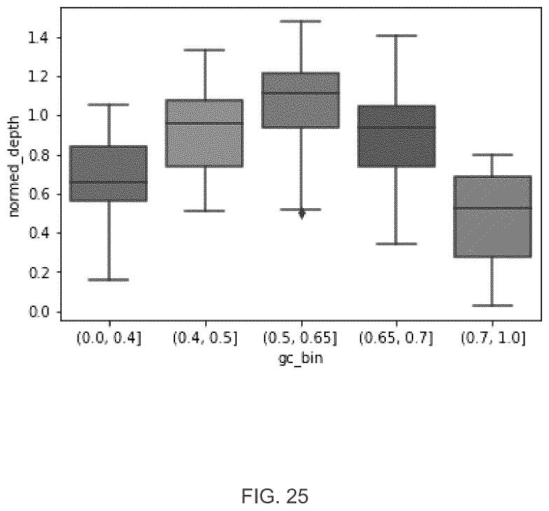

In one embodiment, determining (iii) ease of sequencing/availability of cfDNA depends, at least in part, on the GC content. A skilled artisan would appreciate that other factors contribute to ease of sequencing. Here, relative coverage of genomic regions is reproducible across samples. As such, the consistent (or inconsistent) coverage is used in determining ease of sequencing. FIG. 24 shows sequencing depth (y-axis) compared to GC content for different combinations. As shown in FIG. 25, normalized depth for GC bins (binned according to percent (%) GC content) shows a continuum with normalized depth around 1 having between about 0.4 to about 0.7 percent GC content. This indicates that GC content can serve as a proxy for sequencing depth, whereby a GC content between 0.4 and 0.7 may be optimal for ensuring adequate sequencing depth. This suggests that GC content is a feature value that can be used in the methods described herein to improve sequencing panel assignment.

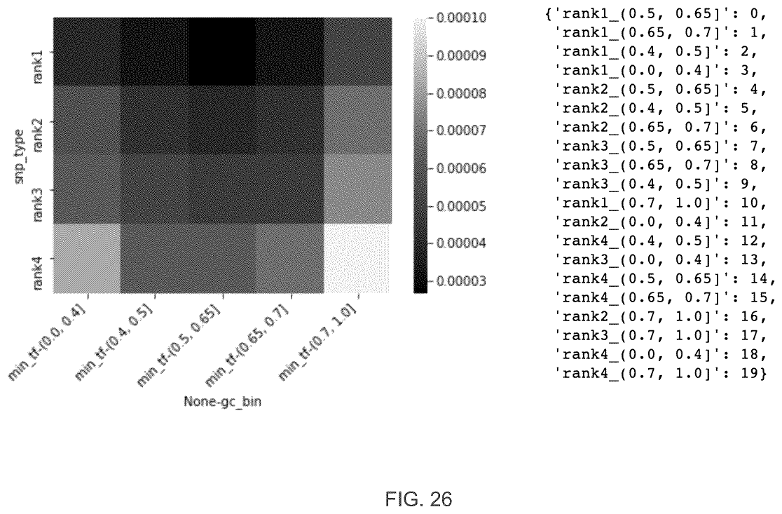

A LoD estimation framework was used to take into account error rates and relative depth in determining variants per participant. For this analysis, 500 sites with the same GC content and error rate, estimated LoD, and rank of combinations based off LoD estimates (GC, SNP type) were used. FIG. 26 shows prioritization of variant (i.e., feature value) selection is driven largely by SNP type (i.e., error rate) except at high GC content. This suggests that SNPs (e.g., excluding those with high error rates) and/or GC content can be used as a feature values in the methods described herein.

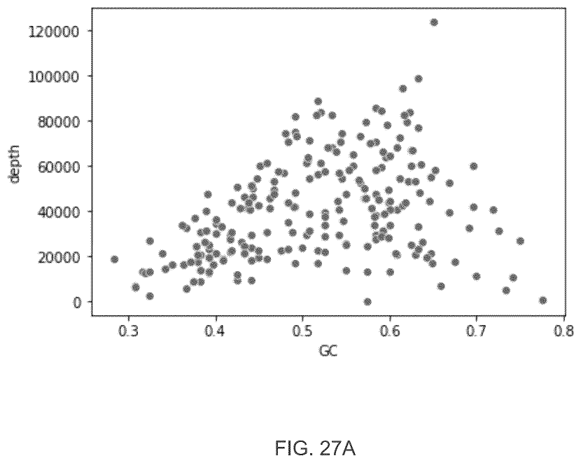

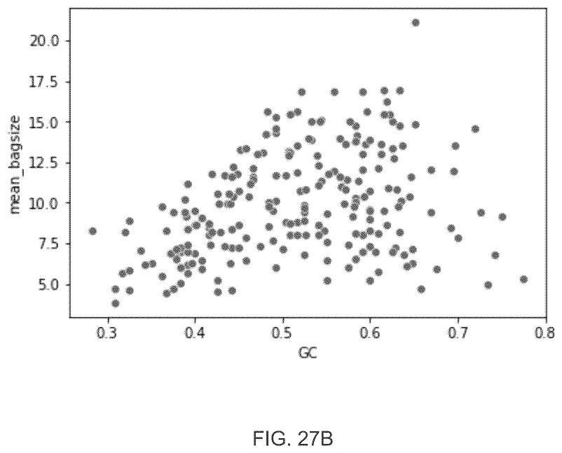

In determining the number of feature values and corresponding genomic regions to include the analysis, per-site (i.e., per genomic region) cost values were determined. In one embodiment, per-site (e.g., per genomic region) cost values depend, at least in part, on the differences between sequencing depth and GC content (see, FIG. 24, FIG. 25, and FIGS. 27A). Comparing depth versus GC content (FIG. 25A to mean_bagsize versus GC content (FIG. 27B) shows that lower coverage sites also have lower bag sizes. This suggests that with a fixed amount of sequencing the relative allocation per target varies by GC content.

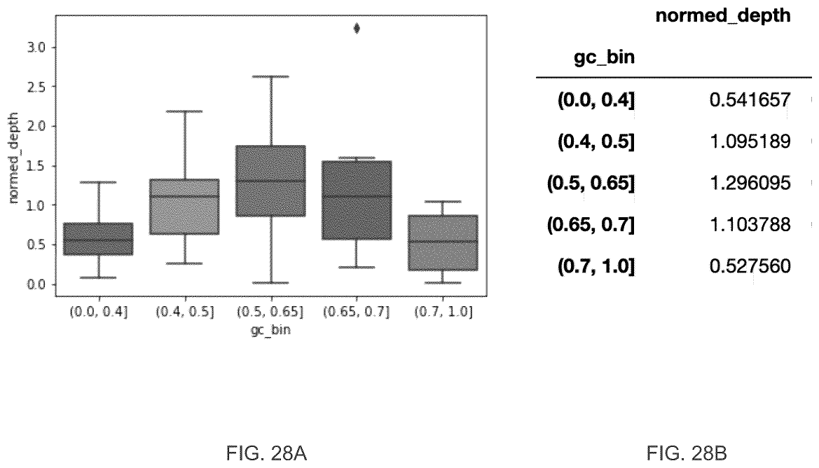

Further analysis for cost per-site (i.e., cost per genomic region) is shown in FIGS. 28A and 28B. There, normalized depth for GC bins (FIG. 28A and 28B) assigns a “relative” cost measure to each genomic region. Raw depth assigned to each region is: total_reads*(target_cost/su(target_costs)). This analysis suggests that GC content between about 0.4 and 0.7 correlates with sequencing depth, and therefore with the relative cost to measure each region. Taken together, samples with GC content between about 0.4 and about 0.7 represent feature values that can be used in the methods described herein.

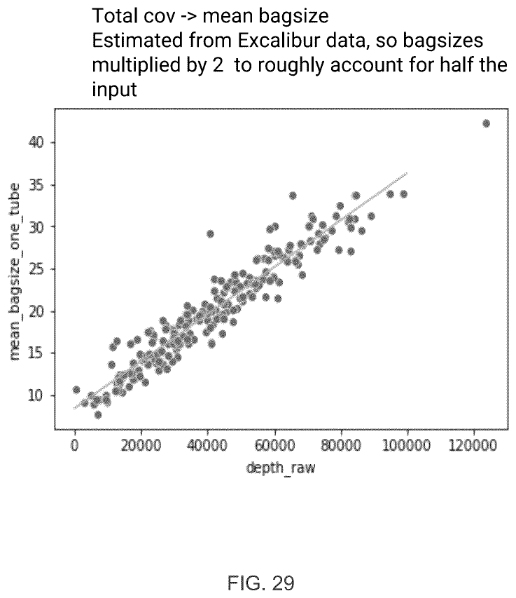

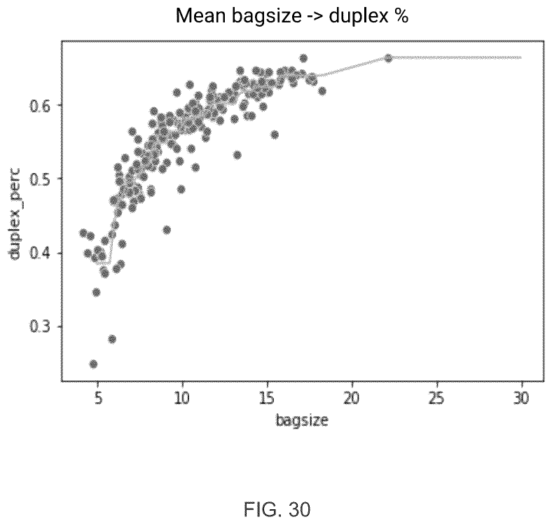

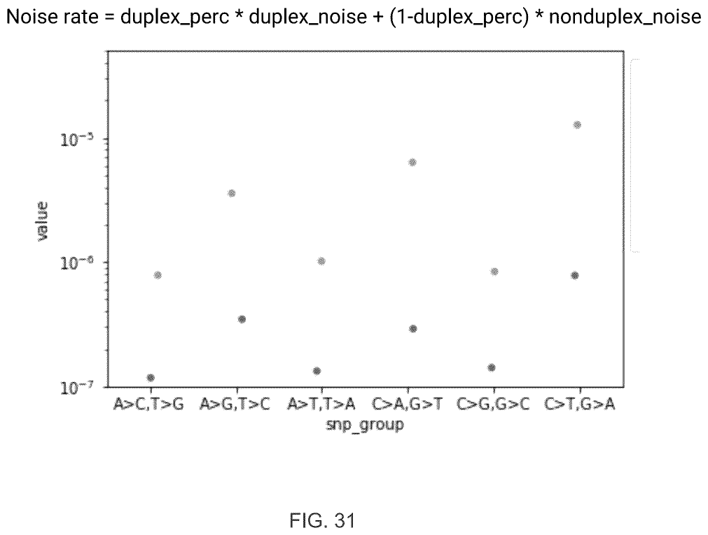

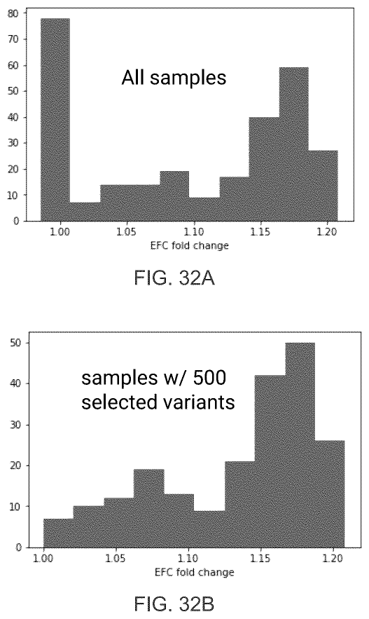

An initial assessment of variant selection looking at relative costs focused on selection with a fixed number of read pairs and total cost. In such cases, factors included: total readpairs per variant (total_rp*(cost/assumed total cost); total coverage (cov) to mean bagsize (and unique cov) (FIG. 29); mean bagsize to duplex % (FIG. 30); duplex % to noise rate (e.g., Noise rate=duplex perc*duplex_noise+(1-duplex_perc)*nonduplex_noise; FIG. 31 (slide 13); and (1-Noise rate)*unique_cov to expected effort free unique coverage (EFC). Variants are then selected based on (EFC/raw RP), until total cost is reached (FIG. 32A and FIG. 32B).

The number of participants (e.g., samples) per panel can be determined empirically. In some embodiments, determining the number of participants per panel includes the sequence cost and panel ordering costs. When sequencing panel assignments include a fixed number of participants, where each participant is assigned to a panel, the number of participants per panel is a tradeoff between the cost of ordering more panels and the increased cost of “wasted” sequencing on participants (i.e., samples) where the requisite information has already been derived.

In one embodiment, the number of participant (i.e., samples) per panel minimizes total cost as a function of the number of panels ordered (with fixed number of participants, number of panels is equivalent to participants per panel). Therefore, minimizing total costs can be represented as:

$$Cost = N * {Cost}_{panel} + {n}_{samples} ( {Cost}_{rp} * depth * sites * \frac{{n}_{samples}}{N} )$$

where N=number of panels, costᵣₚ=cost per read pair, depth=target raw sequencing depth, sites=number of variants per participant, nₛₐₘₚₗₑₛ=total number of participants. Here, the samples are assumed to have the same number of genomic regions per participant. In addition, the per panel cost is assumed to be constant.

As such, the cost of sequencing can be represented as:

$$= \frac{1}{N} ( Cost rp * depth * sites * {n\_samples}^{2} )$$

Here, cost as a function of N is convex for N>0. Solving for N that minimizes overall cost, thereby determining the number of participants per panel can be represented as:

$$N = \sqrt{\frac{{Cost}_{sequencing}}{{Cost}_{panel}}}$$

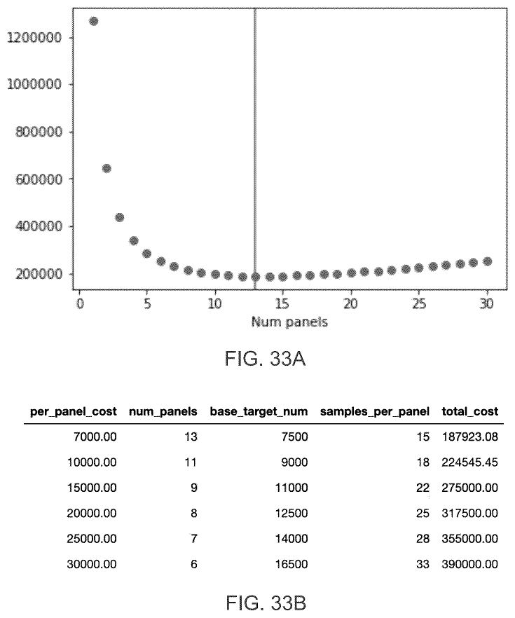

In one example, given a target depth of 35,000x, 500 sites per samples, and 200 samples, the number of participants per panel optimizations are as shown in FIGS. 33A-33B.

### V.B. Early Detection of Cancer

In some embodiments, the methods and/or classifier of the present invention are used to detect the presence or absence of cancer in a subject suspected of having cancer. For example, a classifier (e.g., as described above in Section IV and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e., binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### V.C. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein). The classification may further quantify tumor burden to assess change over time.

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction , then the treatment is considered to have been successful. However, if the second cancer prediction increases compared to the first cancer prediction , then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 50 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 5 months, at least once every 6 months, at least once a year, at least once every 2 years, at least once every 3 years, at least once every 4 years, or at least once every 5 years.

### V.D. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V.E. Kit Implementation

Also disclosed herein are kits for performing the methods described above including the methods relating to the cancer classifier. The kits may include one or more collection vessels for collecting a sample from the individual comprising genetic material. The sample can include blood, plasma, serum, urine, fecal, saliva, other types of bodily fluids, or any combination thereof. Such kits can include reagents for isolating nucleic acids from the sample. The reagents can further include reagents for sequencing the nucleic acids including buffers and detection agents. In one or more embodiments, the kits may include one or more sequencing panels comprising probes for targeting particular genomic regions, particular mutations, particular genetic variants, or some combination thereof. For example, the analytics system may generate differing treatment kits for different targeted sequencing panels as determined with the panel assignment model. In other embodiments, samples collected via the kit are provided to a sequencing laboratory that may use the sequencing panels to sequence the nucleic acids in the sample.

A kit can further include instructions for use of the reagents included in the kit. For example, a kit can include instructions for collecting the sample, extracting the nucleic acid from the test sample. Example instructions can be the order in which reagents are to be added, centrifugal speeds to be used to isolate nucleic acids from the test sample, how to amplify nucleic acids, how to sequence nucleic acids, or any combination thereof. The instructions may further illumine how to operate a computing device as the analytics system 200, for the purposes of performing the steps of any of the methods described.

In addition to the above components, the kit may include computer-readable storage media storing computer software for performing the various methods described throughout the disclosure. One form in which these instructions can be present is as printed information on a suitable medium or substrate, e.g., a piece or pieces of paper on which the information is printed, in the packaging of the kit, in a package insert. Yet another means would be a computer readable medium, e.g., diskette, CD, hard-drive, network data storage, on which the instructions have been stored in the form of computer code. Yet another means that can be present is a website address or QR code which can be used via the internet to access the information at a removed site.

### VI. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1. A method for performing targeted variant sequencing of a set of samples, the method comprising:
obtaining initial sequencing data for each sample describing presence or absence of each of a plurality of genetic variants in a reference genome, wherein the initial sequencing data comprises sequence reads of nucleic acid fragments in a biological sample obtained from a subject;
determining, for each sample, a number of genetic variants present in the sequencing data of the sample;
determining, for each sample, one or more characteristics for each genetic variant present in the sequencing data of the sample;
applying a panel assignment model to determine a panel assignment for each sample to one of a plurality of targeted variant sequencing panels, wherein applying the panel assignment model comprises:
iterating through each sample in the set of samples and to determine the corresponding panel assignment that optimizes uniformity of panel size across the targeted variant sequencing panels and uniformity of characteristics of the genetic variants across samples assigned to each targeted variant sequencing panel,
performing a swapping operation to swap panel assignments of at least two samples to further optimize the uniformity of panel size across the targeted variant sequencing panels and the uniformity of characteristics of the genetic variants across samples assigned to each targeted variant sequencing panel; and
generating each targeted sequencing panel inclusive of samples with panel assignments to the targeted sequencing panel and indicating an aggregate set of genetic variants across the samples assigned to the targeted sequencing panel; and
performing the targeted variant sequencing with each targeted variant sequencing panel comprising the assigned samples on cell-free deoxyribonucleic acid (cfDNA) samples subsequently collected from the subjects.

2. The method of claim 1, wherein the initial sequencing includes whole genome sequencing or whole exome sequencing of each sample.

3. The method of claim 1, wherein the genetic variants include single nucleotide variants, insertion variants, and deletion variants.

4. The method of claim 1, wherein the genetic variants include copy number variants.

5. The method of claim 1, wherein the one or more characteristics for each genetic variant are selected from a group consisting of: a guanine and cytosine content of the genetic variant, an error rate of targeting probes of the genetic variant, a sequencing depth count of the genetic variant, a presence or absence of the genetic variant, a mean allele frequency of the genetic variant, a total number of genetic variants, and an allele frequency of true genetic variants.

6. The method of claim 1, wherein applying the panel assignment model further comprises:
determining the panel assignment for each sample in the set of samples according to one or more hard constraints.

7. The method of claim 6, wherein the one or more hard constraints are selected from a group consisting of: a maximum number of samples per targeted sequencing panel, a maximum number of a samples of one type per targeted sequencing panel, or a total number of genetic variants per targeted sequencing panel.

8. The method of claim 1, wherein the panel assignment model comprises a function scoring the uniformity of panel size across the targeted variant sequencing panels and the uniformity of characteristics of the genetic variants across samples assigned to each targeted variant sequencing panel.

9. The method of claim 8, wherein iterating through each sample in the set of samples to determine the corresponding panel assignment comprises:
applying a greedy algorithm or a dynamic programming algorithm to the function to determine the corresponding panel assignment.

10. The method of claim 8, wherein performing a swapping operation comprises:
evaluating a change in score based on the function evaluating the swap of the at least two samples; and
swapping the panel assignments of the at least two samples based on the change in score being above a threshold.

11. The method of claim 1, wherein the swapping operation is performed iteratively to assess pairs of samples assigned to different targeted sequencing panels.

12. The method of claim 1, further comprising:
for each targeted sequencing panel, identifying an optimal aggregate set of genetic variants across the samples based on the characteristics of the genetic variants in each sample assigned to the targeted sequencing panel.

13. The method of claim 12, wherein identifying the optimal set of genetic variants for each targeted sequencing panel comprises, for each targeted sequencing panel:
determining whether each sample has the total number of genetic variants above a per-sample threshold; and
responsive to determining at least one sample has the total number of genetic variants above the per-sample threshold, identifying a subset of genetic variants in the sample to include in the targeted sequencing panel that optimizes the characteristics of the optimal aggregate set of genetic variants.

14. The method of claim 13, wherein identifying the subset of genetic variants to include in the targeted sequencing panel comprises inclusion of genetic variants present in other samples of the targeted sequencing panel.

15. The method of claim 1, wherein generating each targeted sequencing panel further comprises identifying corresponding targeting probes to target the aggregate set of genetic variants.

16. The method of claim 1, the method further comprising:
obtaining targeted sequencing data for the set of samples from the targeted variant sequencing panels on the cfDNA samples, wherein the targeted sequencing data comprises sequence reads of cell-free nucleic acid fragments in a blood sample;
for each cfDNA sample:
calling one or more variants present in the targeted sequencing data;
determining a feature vector based on the called one or more variants; and
applying a cancer classifier to the feature vector to predict a tumor fraction in the cfDNA sample.

17. (canceled)

18. (canceled)

19. A targeted sequencing panel comprising:
a set of targeting probes to target an aggregate set of genetic variants across a plurality of samples assigned to the targeted sequencing panel, wherein the aggregate set of genetic variants and the plurality of samples are determined by:
obtaining initial sequencing data for each sample in a set of samples describing presence or absence of each of a plurality of genetic variants in a reference genome, wherein the initial sequencing data comprises sequence reads of nucleic acid fragments in a biological sample obtained from one subject;
determining, for each sample, a number of genetic variants present in the sequencing data of the sample;
determining, for each sample, one or more characteristics for each genetic variant present in the sequencing data of the sample;
applying a panel assignment model to determine a panel assignment for each sample to one of a plurality of targeted variant sequencing panels, wherein applying the panel assignment model comprises:
iterating through each sample in the set of samples and to determine the corresponding panel assignment that optimizes uniformity of panel size across the targeted variant sequencing panels and uniformity of characteristics of the genetic variants across samples assigned to each targeted variant sequencing panel,
performing a swapping operation to swap panel assignments of at least two samples to further optimize the uniformity of panel size across the targeted variant sequencing panels and the uniformity of characteristics of the genetic variants across samples assigned to each targeted variant sequencing panel.

20. A plurality of targeted sequencing panels comprising:
for each targeted sequencing panel, a set of targeting probes to target an aggregate set of genetic variants across a plurality of samples assigned to the targeted sequencing panel,
wherein the targeted sequencing panels are determined by:
obtaining initial sequencing data for each sample in a set of samples describing presence or absence of each of a plurality of genetic variants in a reference genome, wherein the initial sequencing data comprises sequence reads of nucleic acid fragments in a biological sample obtained from one subject;
determining, for each sample, a number of genetic variants present in the sequencing data of the sample;
determining, for each sample, one or more characteristics for each genetic variant present in the sequencing data of the sample;
applying a panel assignment model to determine a panel assignment for each sample to one of a plurality of targeted variant sequencing panels, wherein applying the panel assignment model comprises:
iterating through each sample in the set of samples and to determine the corresponding panel assignment that optimizes uniformity of panel size across the targeted variant sequencing panels and uniformity of characteristics of the genetic variants across samples assigned to each targeted variant sequencing panel,
performing a swapping operation to swap panel assignments of at least two samples to further optimize the uniformity of panel size across the targeted variant sequencing panels and the uniformity of characteristics of the genetic variants across samples assigned to each targeted variant sequencing panel.

21-44. (canceled)
